(19) United States
(12) Patent Application Publication
(10) Pub. No.: US 2026/0077071 A1
(43) Pub. Date: Mar. 19, 2026

(54) Targeted Delivery of Theranostic Agents

(71) Applicants: Peter MacCallum Cancer Institute, Melbourne (AU); The University of Melbourne, The University of Melbourne (AU)

(72) Inventors: Mohammad Haskali, Melbourne (AU); Craig A. Hutton, The University of Melbourne (AU)

(21) Appl. No.: 18/847,110

(22) PCT Filed: Mar. 16, 2023
(86) PCT No.: PCT/AU2023/050188

§ 371 Date: Sep. 13, 2024

Related U.S. Application Data
(60) Provisional application No. 63/340,301, filed on May 10, 2022.

(30) Foreign Application Priority Data
Mar. 16, 2022 (AU) 2022900646

(51) Int. Cl. A61K 51/08; C07B 59/00; C07K 7/08

(52) U.S. Cl. CPC A61K 51/088; C07B 59/008; C07K 7/08; A61K 2121/00; A61K 2123/00; C07B 2200/05

(57) ABSTRACT

The present invention relates to compounds and methods for the targeted delivery of diagnostic/therapeutic radionuclides to cancer tissue. In particular, the present invention relates to the targeted delivery of diagnostic/therapeutic radionuclides to cholecystokinin-2 receptor positive cancers and to methods for the diagnosis and treatment of cancer.

### STATEMENT REGARDING SEQUENCE LISTING

This application contains a Sequence Listing, which has been submitted electronically in xml format and is hereby incorporated by reference in its entirety. Said xml copy, created on Dec. 1, 2025, is named 35621511.xml.xml and is 150,921 bytes in size.

### FIELD OF THE INVENTION

The present invention relates to compounds and methods for the targeted delivery of diagnostic/therapeutic radionuclides to cancer tissue. In particular, the present invention relates to the targeted delivery of diagnostic/therapeutic radionuclides to cholecystokinin-2 receptor positive cancers and to methods for the diagnosis and treatment of cancer.

### BACKGROUND OF THE INVENTION

The fundamental principle underlying the field of theranostic radiopharmaceuticals is the development of targeted patent compounds (such as short peptides) that function as therapeutic or diagnostic compounds contingent upon the radionuclide complexed to them.

This rapidly developing field of personalised medicine has already proven highly successful in treating patients with metastatic malignancies with striking efficacy and low toxicity (Hofman, M. S., et al., Lancet, 2020, 395(10231), 1208-1216; Hofman, M. S. P., et al., Lancet Oncology, 2018, 19(6), 825-833; Kaltsas, G., et al., Neuroendocrinology 2017, 105(3), 245-254). These remarkable outcomes are due in part to the unique ability for clinicians to “see what they treat”. Moreover, the increased sensitivity and specificity of these compounds allows clinicians to more accurately stage patients before, during and after treatment to ensure patients receive the most appropriate disease management (Velikyan, I. Pharmaceuticals (Basel), 2020, 13, 39). The implementation of peptide-based theranostics in oncology is currently limited to only a few cancers. These include somatostatin-2R positive neuroendocrine tumours and prostate specific membrane antigen (PSMA) positive prostate cancer.

The G-protein coupled receptor cholecystokinin-2 (CCK-2R), whose natural ligand is the gastrin hormone G17, is an important molecular target for theranostic application that is overexpressed on a range of cancers including, but not limited to, medullary thyroid carcinoma (MTC), somatostatin-2R negative neuroendocrine tumours (Gotthardt, M., et al., Endocrine-Related Cancer, 2006, 13(4), 1203-1211; Reubi, J. C., Schaer, J. C., Waser, B., Cancer Research, 1997, 57(7), 1377-1386; Behe, M., Behr, T. M., Peptide Science 2002, 66(6), 399-418; Reubi, J. C., Maecke, H. R., Journal of Nuclear Medicine 2017, 58 (Supplement 2), 10S-16S), stromal ovarian cancer (100% incidence; Reubi, J. C., Schaer, J. C., Waser, B., Cancer Research, 1997, 57(7), 1377-1386), and small cell lung cancer (56% incidence; Roy, J., et al., Oncotarget 2016, 7(12), 14605-14615; Reubi, J. C., Macke, H. R., Krenning, E. P., Society of Nuclear Medicine 2005, 46Suppl 1, 67s-75s; Copps, J., Murphy, R. F., Lovas, S., Protein Pept Lett 2009, 16 (12), 1504-1518; Zeng, Q., et al., Frontiers in Endocrinology 2020, 11(112)), among many others.

Given the well-established link between CCK-2R and cancer, much research has already been done to understand the critical features of the G17 peptide in an attempt to establish a modified CCK-2R peptide suitable for use as a theranostic radiopharmaceutical in nuclear medicine (Roosenburg, S., et al., Amino Acids, 2010, 41, 1049-1058). Several radiolabelled gastrin derived peptide analogues have been developed, including DGlu-MG0 derived from human minigastrin, the truncated form MG11, missing the penta-glutamic acid sequence and other derivatives of gastrin and cholecystokinin (Rottenburger, C., et al., J. Nucl. Med., 2020, 61, 520-526; Erba, P. A., et al., Pol. Arch. Intern. Med., 2018, 128, 791-795; Klinger, M., et al., Journal of Nuclear Medicine 2018). CP04 and its closely related analogue PP-F11N containing an N-terminal hexa-D-glutamic acid sequence, exhibit reduced kidney uptake and retention (minimal nephrotoxicity) and enhanced metabolic stability, desirable traits required for a theranostic compound. Both CP04 and PP-F11N have been investigated in recent clinical trials for imaging and targeting CCK-2R in humans (Rottenburger, C., et al., J. Nucl. Med., 2020, 61, 520-526; Erba, P. A., et al., Pol. Arch. Intern. Med., 2018, 128, 791-795). Despite a demonstrated accumulation in MTC lesions for both peptides, a suboptimal tumour radiation dose was observed in comparison to [¹⁷Lu]Lu-DOTA-TATE, used in the treatment of neuroendocrine tumours, indicating that improved agents are still needed. High tumour uptake and long residence time are of paramount importance for successful delivery of Peptide Receptor Radionuclide Therapy (PRRT) whereby they determine the absorbed dose to the tumour.

von Guggenberg and co-workers developed the peptide MGS5 using unnatural aromatic amino acids and N-methylation of residues at the C-terminus of MG11. Radiolabelled MGS5 displayed improved in vitro and in vivo characteristics with enhanced metabolic stability and improved tumour targeting when compared to CP04 and PP-F11N (Klinger, M., et al., Journal of Nuclear Medicine 2018; Klinger, M., et al., Journal of Nuclear Medicine 2019, 60(1), 1010-1016; Klinger, M., et al., Theranostics, 2018, 8, 2896-2908; Maximilian, K., Anton Amadeus, H., Guggenberg, E. v., Current Medicinal Chemistry, 2020, 27, 1-21). The clinical translation of [⁶⁸Ga]Ga-DOTA-MG5S arising from these studies has recently been reported in two patients, demonstrating the concept of CCK-2R PET imaging in patients with MTC (Guggenberg, E. v., et al., Eur. J Nucl. Med. Mol. Imaging, 2019, 46 (Si), S268; Uprimny, C., et al., Eur. J Nucl. Med. Mol. Imaging, 2020).

Until now, only a limited number of studies exist elucidating the impact amino acid modifications have upon conformation of radiolabelled G17-based CCK-2R binding ligands (CP04, PP-F11N and MGS5), and thus receptor binding and biological activity (Kolenc Peitl, P., et al., Bioconjugate Chemistry, 2015, 26, 1113-1119). Although the critical sequence required for the peptide-CCK-2R interaction is known to be the C-terminal tetra-peptide Trp-Met-Asp-Phe-NH₂, this accounts for only 10% to the total biological potency (measured by gastric acid secretion in rats) of G17. Interestingly, gradual inclusion of polyglutamate residues at the N-terminus results in conformational change, as evidenced by CD spectroscopy, and incrementally increases the biological potency to 90% (Peggion, E., Foffani, M. T., Mammi, S., Structure-Function Relationship of Gastrin Hormones: A Rational Approach to Drug Design. In Bioorganic Chemistry in Healthcare and Technology, Pandit, U. K.; Alderweireldt, F. C., Eds. Springer US: Boston, MA, 1991, 121-133). Peggion et al. concluded that although the N-terminus of G17 likely adopts an α-helical conformation, an interaction between the “head” and the “tail” of the peptide must exist to explain the increasing potency and the conformational changes observed upon pentaglutamyl sequence elongation. It was suggested that a β-turn centred on residues Ala¹¹-Trp¹⁴ is likely to exist, capping the α-helical section of the N-terminus and bringing both ends of the peptide in close proximity. This study highlighted the pharmacological significance of a suspected “U-shaped” hairpin fold, with the α-helical N-terminus stabilizing the biologically active conformation of the C-terminus (Copps, J., Murphy, R. F., Lovas, S., Protein Pept. Lett., 2009, 16, 1504-1518).

The three-dimensional structure of any receptor ligand is clearly important for binding specificity, affinity and functionality and should be considered during the design and optimisation of any targeting peptide intended as a theranostic compound. However, the design of short-medium peptides with defined secondary structure is difficult to implement as they are flexible in nature and unlikely to form ordered structures akin to those found in larger proteins and biomolecules. The development of targeting peptides requires detailed mimicking of the interproteinic contact points, which often necessitate the recapitulation of not only the biomacromolecular primary structure but also the secondary structure. Peptide cyclisation and stapling have been used to restrict peptide conformations through the formation of covalent bonds. However, cyclisation and stapling do not give rise to an array of secondary structures and are less useful when a linear peptide is needed for functionality.

Accordingly, there exists a need to develop new theranostic compounds to expand their transformative role in managing other malignancies.

### SUMMARY OF THE INVENTION

New compounds and methods are provided for diagnosing and treating cancer in a subject. Accordingly, in one aspect the present invention provides a compound of Formula (I):

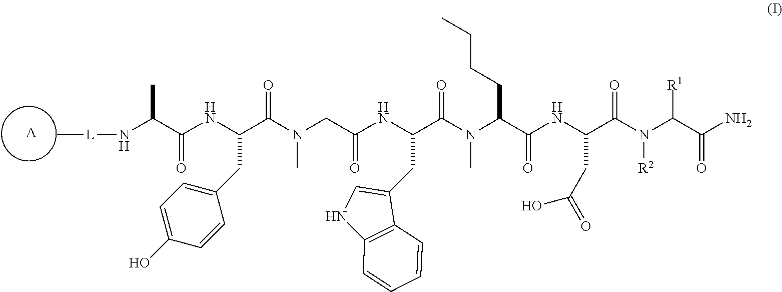

wherein
R¹ is a side chain of an amino acid selected from phenylalanine, 3-(1-naphthyl)alanine, 3-(2-naphthyl)alanine, or tyrosine, wherein the phenylalanine residue is optionally substituted with one or more halo, —NH₂, cyano, C₁₋₆alkyl, haloC₁₋₆alkyl, or acetyl;
R² is selected from H or C₁-C₃alkyl;
A is a radionuclide binding ligand; and
-L- is an amino acid sequence: -D-Glu-D-Glu-D-Ala-D-Glu-D-Glu-D-Glu- (SEQ ID NO: 32); or
a pharmaceutically acceptable salt thereof.

In one embodiment, the compound of Formula (I) is represented by Formula (Ia):

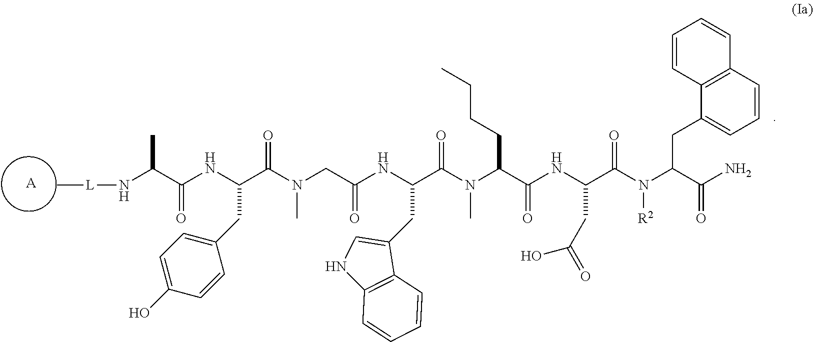

In another embodiment, the compound of Formula (I) is represented by Formula (Ib):

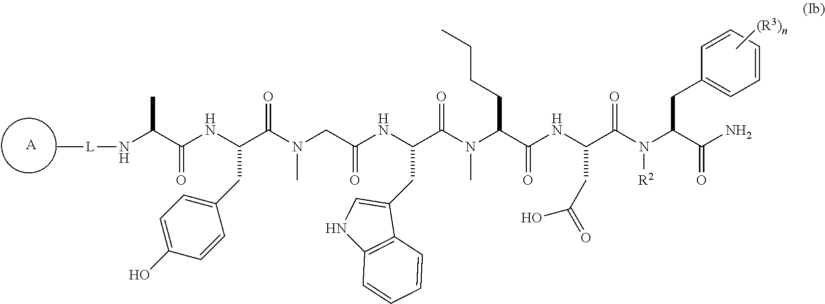

wherein each occurrence of R³ is independently selected from halo, —NH₂, cyano, —OH, C₁₋₆alkyl, haloC₁₋₆alkyl, or acetyl; and n is from 0 to 5.

In a further embodiment, the compound of Formula (I) is represented by Formula (Ic):

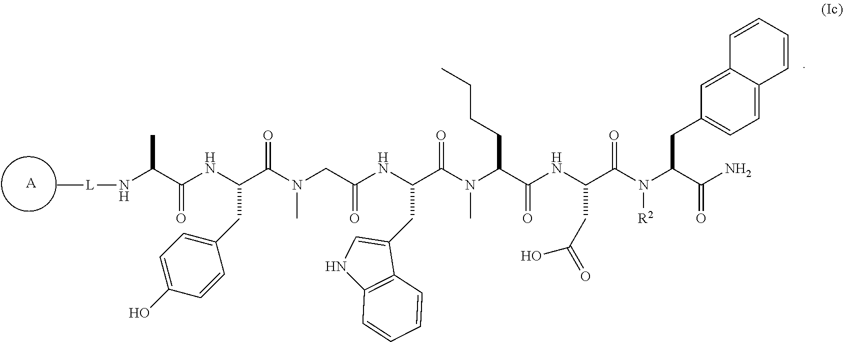

In one embodiment, the compounds according to the invention further comprise a radionuclide complexed to the radionuclide binding ligand.

In another aspect, the present invention provides a method for identifying CCK-2R positive cancer in a subject, comprising administering to the subject an effective amount of a compound according to the invention, or a pharmaceutically acceptable salt thereof, and detection of the radionuclide.

In a further aspect, the invention provides a compound according to the invention, or a pharmaceutically acceptable salt thereof, for use in a method of identifying CCK-2R positive cancer in a subject, the method comprising administering to the subject an effective amount of said compound and detection of the radionuclide.

In another aspect there is provided a method for treating CCK-2R positive cancer in a subject in need thereof, comprising administering to the subject a compound according to the invention, or a pharmaceutically acceptable salt thereof.

In yet another aspect of the invention there is provided a pharmaceutical composition comprising a compound according to the invention, or a pharmaceutically acceptable salt thereof.

These and other aspects of the present invention will become more apparent to the skilled addressee upon reading the following detailed description in connection with the accompanying examples and claims.

### BRIEF DESCRIPTION OF THE DRAWINGS

The invention will herein be described by way of example only with reference to the following non-limiting Figures in which:

FIG. 1 illustrates a graphical representation of the binding curves for IC₅₀ determination of Peptides A-F, comparative example-1 and CP04 against [¹⁷⁷Lu]Lu-DOTA-CP04 in A431-CCK2R cells. Curves are representative of 2-4 biological replicates performed in three technical replicates (n=3).

FIG. 2 illustrates a graphical representation of the binding curves for IC₅₀ determination of Compounds 1-4 and comparative example-2 against [¹⁷⁷Lu]Lu-DOTA-CP04 in A431—CCK2R cells. Curves are representative of 2-4 biological replicates performed in three technical replicates (n=3).

FIG. 3 illustrates a graphical representation of the CD spectra of Compounds ^{nat}Ga-1-4 and ^{nat}Ga-Cmp ex-2 in water and 5 mM DPC micelles. Three spectra accumulated at 20° C. were averaged and baseline corrected.

FIG. 4 illustrates a graphical representation of metabolic stability of Compounds [⁶⁸Ga]Ga-1-4 and [⁶⁸Ga]Ga-Cmp ex-2 incubated in human serum, liver homogenates, and kidney homogenates. Samples were assayed at various time points over 90 min. The results are mean values (n=2).

FIG. 5 illustrates a) distribution of Compounds [⁶⁸Ga]Ga-1-4 and [⁶⁸Ga]Ga-Cmp ex-2 in Female BALB/c nu/nu mice (age 8-10 weeks). b) Tumour uptake analysis of Compounds [⁶⁸Ga]Ga-1-4 and [⁶⁸Ga]Ga-Cmp ex-2 at 1 hr, 2.5 hr post injection and with co-administration of the blocking agent YM022, reported as % ID/g. c) Quantification analysis of Compounds [⁶⁸Ga]Ga-1-4 and [⁶⁸Ga]Ga-Cmp ex-2 accumulated in selected organ at 1 hr, reported as % ID/g. d) Quantification analysis presenting tumour-to-organ ratio of Compounds [⁶⁸Ga]Ga-1-4 and [⁶⁸Ga]Ga-Cmp ex-2 at 1 hr post injection. T:B; tumour-to-blood, T:Stom; tumour-to-stomach.

### DETAILED DESCRIPTION OF THE INVENTION

Foldamers provide several advantages including the ability to design secondary structures, control over the orientation of side-chain functional groups and resistance towards proteolytic degradation. Moreover, foldamers address the thermodynamic basis of protein-protein interactions (PPIs) by minimising conformational degrees of freedom, thereby minimising the entropic penalty paid upon binding and resulting in higher binding affinities (Du, X., et al., International Journal of Molecular Sciences 2016, 17(2), 144). Foldamers adopt inherent propensity to form secondary and sometimes tertiary structures through hydrogen bonding, giving rise to a wide array of conformations including β-turns in all its forms, hairpin structures and helices.

In one embodiment, the present invention provides compound of Formula (I):

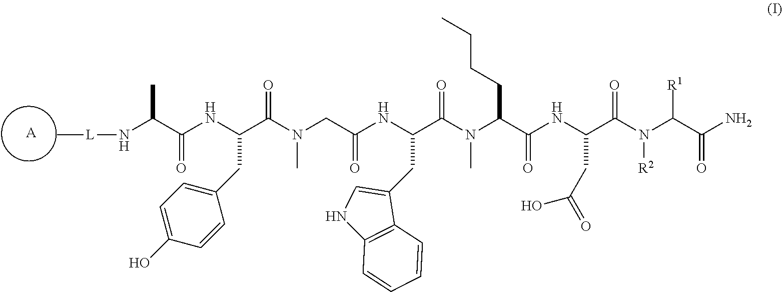

wherein
R¹ is a side chain of an amino acid selected from phenylalanine, 3-(1-naphthyl)alanine, 3-(2-naphthyl)alanine, or tyrosine, wherein the phenylalanine residue is optionally substituted with one or more halo, —NH₂, cyano, C₁₋₆alkyl, haloC₁₋₆alkyl, or acetyl;
R² is selected from H or C₁-C₃alkyl;
A is a radionuclide binding ligand; and
-L- is an amino acid sequence: -D-Glu-D-Glu-D-Ala-D-Glu-D-Glu-D-Glu- (SEQ ID NO: 32); or
a pharmaceutically acceptable salt thereof.

Substituting the amino acid residue to the N-terminal side of the C-terminal tetra-peptide Trp-Met-Asp-Phe-NH₂ with the turn-inducing residue N-methylglycine was found to improve binding affinity of the resultant compounds. It is believed that inclusion of N-methylglycine constrains the compounds in a β-hairpin conformation. The structure is believed to be further stabilised by interactions between the aromatic and glutamic acid side chains.

In another embodiment, the invention provides a peptide of Formula (II):

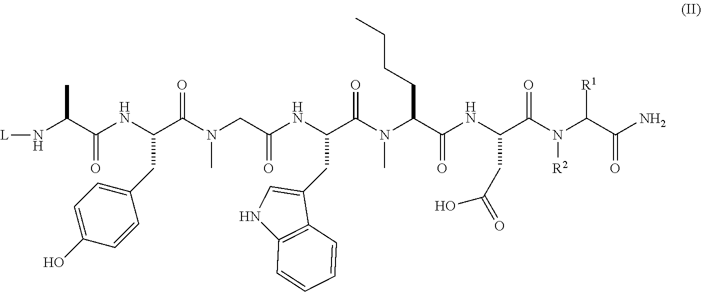

wherein
R¹ is a side chain of an amino acid selected from phenylalanine, 3-(1-naphthyl)alanine, 3-(2-napthylalanine), or tyrosine, wherein the phenylalanine residue is optionally substituted with one or more halo, —NH₂, cyano, C₁₋₆alkyl, haloC₁₋₆alkyl, or acetyl;
R² is selected from H or C₁-C₃alkyl; and
-L- is an amino acid sequence: -D-Glu-D-Glu-D-Ala-D-Glu-D-Glu-D-Glu- (SEQ ID NO: 32); or
a pharmaceutically acceptable salt thereof.

In one embodiment, the peptide of Formula (II), or the pharmaceutically acceptable salt thereof, is represented by Formula (IIa):

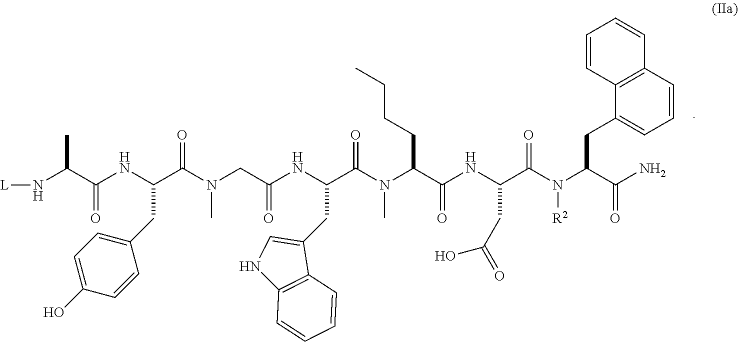

In another embodiment, the peptide of Formula (II), or the pharmaceutically acceptable salt thereof, is represented by Formula (IIb):

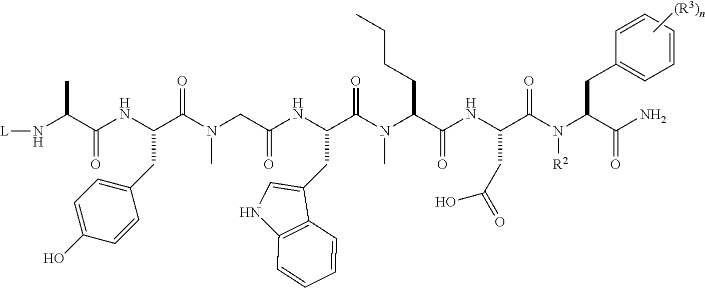

wherein each occurrence of R³ is independently selected from halo, —NH₂, cyano, —OH, C₁₋₆alkyl, haloC₁₋₆alkyl, or acetyl; and n is from 0 to 5.

In a further embodiment, the peptide of Formula (II), or the pharmaceutically acceptable salt thereof, is represented by Formula (IIc):

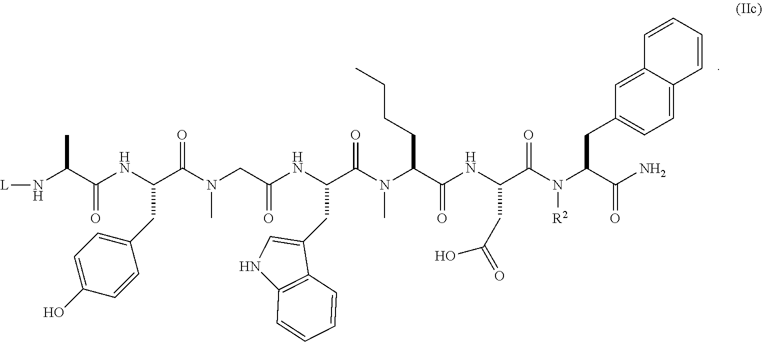

In another embodiment, the invention provides a peptide of Formula (II) selected from those listed in Table 1.

TABLE 1
Peptides of Formula (II)

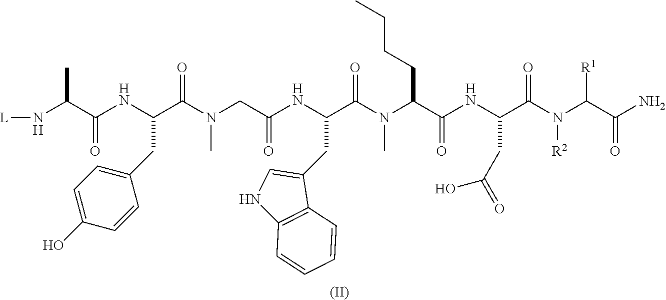

| -L- | R¹ | R² | SEQ ID NO: |
|---|---|---|---|
| -(D-Glu)₂-D-Ala-(D-Glu)₃- | Nal¹ | H | 1 |
| -(D-Glu)₂-D-Ala-(D-Glu)₃- | Phe | methyl | 2 |
| -(D-Glu)₂-D-Ala-(D-Glu)₃- | Phe | H | 3 |
| -(D-Glu)₂-D-Ala-(D-Glu)₃- | Nal | Methyl | 4 |
| -(D-Glu)₂-D-Ala-(D-Glu)₃- | Phe(4-F)² | H | 5 |
| -(D-Glu)₂-D-Ala-(D-Glu)₃- | Phe(3,4-diF)³ | H | 6 |
| -(D-Glu)₂-D-Ala-(D-Glu)₃- | Phe(3,4,5,-F)⁴ | H | 7 |
| -(D-Glu)₂-D-Ala-(D-Glu)₃- | Phe(4-NH₂)⁵ | H | 8 |
| -(D-Glu)₂-D-Ala-(D-Glu)₃- | 2-Nal⁶ | H | 9 |
| -(D-Glu)₂-D-Ala-(D-Glu)₃- | Phe(4-CN)⁷ | H | 10 |
| -(D-Glu)₂-D-Ala-(D-Glu)₃- | Phe(4-CF₃)⁸ | H | 11 |
| -(D-Glu)₂-D-Ala-(D-Glu)₃- | Tyr | H | 12 |
| -(D-Glu)₂-D-Ala-(D-Glu)₃- | Phe(2,3,4,5,6-F)⁹ | H | 13 |
| -(D-Glu)₂-D-Ala-(D-Glu)₃- | Phe(3-F)¹⁰ | H | 14 |
| -(D-Glu)₂-D-Ala-(D-Glu)₃- | Phe(4-Ac)¹¹ | H | 15 |

¹Nal = 3-(1-naphthyl)-L-alanine;
²Phe(4-F) = 4-fluoro-L-phenylalanine;
³Phe(3,4-diF) = 3,4-difluoro-L-phentylalanine;
⁴Phe(3,4,5-F)⁴ = 3.4.5-trifluoro-L-phenylalanine;
⁵Phe(4-NH₂) = 4-amino-L-phenylalanine;
⁶2-Nal = 3-(2-naphthyl)-L-alanine;
⁷Phe(4-CN) = 4-cyano-L-phenylalanine;
⁸Phe(4-CF₃) = 4-trifluoromethyl)-L-phenylalanine;
⁹Phe(2,3,4,5,6-F) = 2,3,4,5,6-pentafluoro-L-phenylalanine;
¹⁰Phe(3-F) = 3-fluoro-L-phenylalanine
¹¹Phe(4-Ac) = 4-(acetyl)-L-phenylalanine.

Reference to an amino acid “side chain” takes its standard meaning in the art. Examples of side chains of amino acids are shown below:

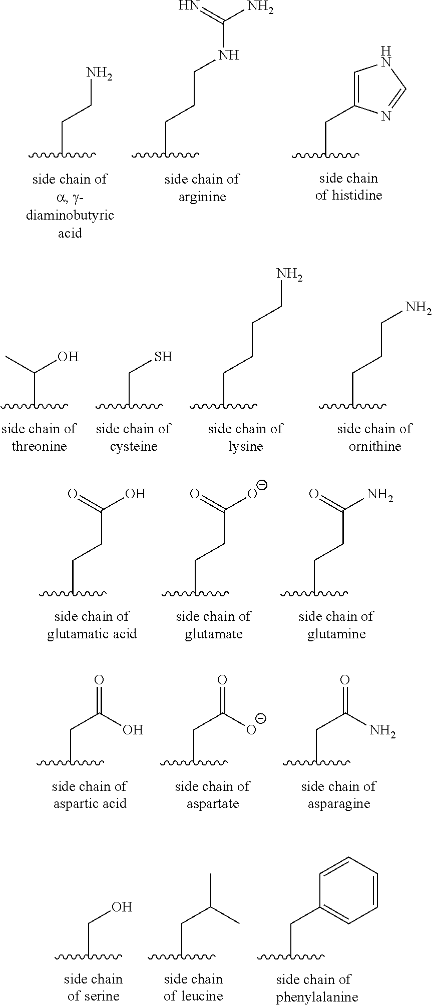

As used herein, non-naturally occurring amino acids include any compound with both amino and carboxyl functionality, derivatives thereof, or derivatives of a naturally occurring amino acid. These amino acids form part of the peptide chain through bonding via their amino and carboxyl groups. Alternatively, these derivatives may bond with other natural or non-naturally occurring amino acids to form a non-peptidyl linkage.

In addition to the negatively charged side chains shown above, it will be appreciated that a number of the side chains may also be protonated and so become positively charged, such as the side chain of lysine. The present invention contemplates within its scope these protonated side chains as well.

It will be understood that the compounds of the present invention may exist in one or more stereoisomeric forms (e.g. diastereomers). The present invention includes within its scope all of these stereoisomeric forms either isolated (in, for example, enantiomeric isolation), or in combination (including racemic mixtures and diastereomic mixtures). The present invention contemplates the use of amino acids in both L and D forms, including the use of amino acids independently selected from L and D forms, for example, where the compound comprises two Glu residues, each Glu residue may have the same, or opposite, absolute stereochemistry.

The compounds of the invention may further comprise a radionuclide complexed or covalently bound to the radionuclide binding ligand. As mentioned above, the compounds of the invention are useful as theranostic compounds in that the same peptide may be covalently coupled to a radionuclide binding ligand that is suitable for binding radionuclides useful for diagnosis of cancer and/or a radionuclide binding ligand that is suitable for binding radionuclides useful for the treatment of cancer. In one embodiment, it is envisaged that the radionuclide binding ligand that is suitable for binding radionuclides useful for the treatment of cancer is the same as the radionuclide binding ligand suitable for binding radionuclides useful for diagnosis of cancer. In a further embodiment, the radionuclide binding ligand that is suitable for binding radionuclides for the treatment of cancer differs from the radionuclide binding ligand suitable for binding radionuclides for diagnosis of cancer.

Reference to “a radionuclide binding ligand” will be understood to mean a ligand or chelator that tightly binds a radionuclide (radioisotope). The radionuclide binding ligand is covalently bound to the compound so that, when a compound of the invention is administered to a subject, the compound can deliver the radionuclide to the target site without, or with minimal, radionuclide loss, effectively supplying a site-specific radioactive source in vivo for imaging or therapy. Examples of suitable radionuclide binding ligands include, but are not limited to, DOTA (1,4,7,10-tetraazacyclododecanel,4,7,10-tetraacetic acid), DOTA-NHS-ester, p-SCN-Bn-DOTA (C-DOTA), DOTAGA, DOTAGA-anhydride, CB-DO2A (4,10-bis(carboxymethyl)-1,4,7,10-tetraazabicyclo[5.5.2]tetradecane), TCMC (1,4,7,10-tetrakis(carbamoylmethyl)-1,4,7,10-tetraazacyclododecane), p-SCN-Bn-TCMC, 3p-C-DEPA (2-[(carboxymethyl)]-[5-(4-nitrophenyl-1-[4,7,10-tris-(carboxymethyl)-1,4,7,10-tetraazacyclododecan-1-yl]pentan-2-yl)-amino]acetic acid), 3p-C-DEPA-NCS, p-NH₂-Bn-Oxo-DO3A, TETA (1,4,8,11-tetraazacyclotetradecanel,4,8,11-tetraacetic acid), BAT, p-NH₂-Bn-TE3A, C-TETA, CB-TE2A (4,11-bis-(carboxymethyl)-1,4,8,11-tetraazabicyclo[6.6.2]-hexadecane), CB-TE1A1P, CB-TE2P, MM-TE2A, DM-TE2A, TE2A, Diamsar, SarAr (1-N-(4-Aminobenzyl)-3,6,10,13,16,19-hexaazabicyclo[6.6.6]-eicosane-1,8-diamine), AmBaSar, BaBaSar, NOTA (1,4,7-triazacyclononane-1,4,7-triacetic acid), p-SCN-Bn-NOTA, NODASA, NODAGA, NETA ({4-[2-(bis-carboxymethylamino)-ethyl]-7-carboxymethyl-[1,4,7]triazonan-1-yl}-acetic acid), NETA-monoamide, C-NE3TA-NCS, C-NETA-NCS, 3p-C-NETA, TACN-TM (N,N′,N″-tris(2-mercaptoethyl)-1,4,7-triazacyclononane), DTPA (diethylenetriaminepentaacetic acid), p-SCN-Bn-1B-DTPA, p-SCN-Bn-1B4M-DTPA, CHX-A″-DTPA (2-(p-isothiocyanatobenzyl)-cyclohexyldiethylenetriaminepentaacetic acid), p-SCN-Bn-CHX-A″-DTPA, TRAP (1,4,7-triazacyclononanel,4,7-tris[methyl(2-carboxyethyl)phosphinic acid]), AAZTA (1,4-bis(hydroxycarbonyl methyl)-6-[bis(hydroxylcarbonyl methyl)]amino-6-methyl perhydro-1,4-diazepine), NOPO, H2dedpa (1,2-[[6-(carboxy)-pyridin-2-yl]-methylamino]ethane), H4octapa (N,N′-bis(6-carboxy-2-pyridylmethyl)-ethylenediamine-N,N′-diacetic acid), H2azapa (N,N′-[1-benzyl-1,2,3-triazole-4-yl]methyl-N,N′-[6-(carboxy)pyridin-2-yl]-1,2-diaminoethane), H5decapa (N,N″-[[6-(carboxy)pyridin-2-yl]methyl]-diethylenetriamine-N,N′,N″-triacetic acid), p-SCN-Bn-H40ctapa, HBED (N,N′-bis(2-hydroxybenzyl)-ethylenediamine-N,N′-diacetic acid), HBED-CC, (HBED-CC)TFP, SHBED (N,N′-bis(2-hydroxy-5-sulfobenzyl)-ethylenediamine-N,N′-diacetic acid), BPCA, CP256 (4-acetylamino-4-[2-[(3-hydroxy1,6-dimethyl-4-oxo-1,4-dihydro-pyridin2-ylmethyl)-carbamoyl]-ethyl]-heptanedioic acid bis-[(3-hydroxy-1,6-dimethyl4-oxo-1,4-dihydro-pyridin-2-ylmethyl)-amide]), PCTA (3,6,9,15-tetraazabicyclo[9.3.1]-pentadeca-1(15),11,13-triene-3,6,9,-triacetic acid), p-SCN-Bn-PCTA, DFO, p-SCN-Bn-DFO, H₆phospha (N,N′-(methylenephosphonate)-N,N′-[6-(methoxycarbonyl)pyridin-2-yl]-methyl-1,2-diaminoethane), HEHA (1,4,7,10,13,16-hexaazacyclohexadecane-N,N′,N″,N′″,N″″,N″″″-hexaacetic acid), p-SCN-Bn-HEHA, PEPA (1,4,7,10,13-pentaazacyclopentadecane-N,N′,N″,N′″,N″″-pentaacetic acid), p-SCN-Bn-PEPA, Crown, MACROPA (4-amino-6-[[16-[(6-carboxypyridin-2-yl)mnethyl]-1,4,10,13-telraoxa-7,16-diazacyclooctadec-7-yl]methyl]pyridine-2-carboxylic acid), MACROPA-NCS, pypa, py4pa (6,6′-(((azanediylbis(ethane-2,1-diyl))bis((carboxymethyl)azanediyl))bis(methylene)) dipicolinic acid), noneunpa (6,6′-(((oxybis(ethane-2,1-diyl))bis((carboxymethyl)azanediyl))bis (methylene))dipicolinic acid), DOTAM (2,2′,2″,2′″-(1,4,7,10-tetraazacyclododecane-1,4,7,10-tetrayl)tetraacetamide), and derivatives thereof. In one embodiment, the radionuclide binding ligand is DOTA.

Alternatively, the radionuclide binding ligand may be a peptide or small organic moiety to which the radionuclide is covalently bound. As an example, the radionuclide binding ligand may be a short peptide or organic moiety with a radionuclide such as flourine-18, iodine-124 or iodine-131 covalently bound at an appropriate location. It will be understood that the radionuclide binding ligand should be selected such that it does not compete with the compound at the binding site at the CCK-2 receptor.

In one embodiment, the present invention provides a method for identifying CCK-2R positive cancer in a subject comprising administering to the subject an effective amount of a compound according to the invention, or a pharmaceutically acceptable salt thereof, and detection of the radionuclide.

In another embodiment, the present invention provides a compound according to the invention, or a pharmaceutically acceptable salt thereof, for use in a method of identifying CCK-2R positive cancer in a subject, the method comprising administering to the subject an effective amount of said compound and detection of the radionuclide.

Compounds according to the present invention localise to cancers that overexpress CCK-2 receptors such as medullary thyroid carcinoma (MTC), somatostatin-2R negative neuroendocrine tumours, stromal ovarian cancer (100% incidence) and small cell lung cancer (56% incidence).

After administration of the compound, for example, by intravenous injection, the subject is placed on the scanner. As the injected radionuclide decays it emits a positron that annihilates with an electron, producing a pair of gamma rays or photons that travel in opposite directions. In general terms, the emitted photons are detected when they reach a scintillator material in the scanning device, creating a burst of light that is detected by photomultiplier tubes. Suitable scanning methods include computed tomography (CT), positron emission tomography (PET) and the combined procedure PET-CT.

Radionuclides for diagnosis that are within the scope of the invention include, but are not limited to, carbon-11, nitrogen-13, oxygen-15, fluorine-18, scandium-44, copper-64, gallium-67, gallium-68, yttrium-86, zirconium-89, technetium-99m, indium-111, iodine-124, iodine-125 and terbium-152.

In one aspect, the compound of the invention may be administered in the application of tumour ablation therapies to detect the extent of damage occurring in the affected tissue. The compound localizes to the CCK-2R positive tumour and indicates to a medical practitioner the tumour size and location and, in turn, allows for the continuous monitoring to track tumour size and indicate the effectiveness of a medical treatment method. The ability to monitor the effectiveness of an ongoing therapeutic treatment allows a subject to avoid undergoing ineffective medical treatment and, in turn, helps to develop patient-specific therapy. This is of particular value in fields where a wide variety of potential therapeutics are available, for example, in cancer treatment a wide number of chemo-therapeutics and radio-therapeutics are available. Continually monitoring tumour size through the use of the compounds allows for an earlier assessment of the effectiveness of a particular therapy and, in turn, allows a subject to avoid prolonged exposure to an ineffective line of treatment. The ability of the compound to indicate the ineffectiveness of a medical treatment enables a medical practitioner to alter or change a course of medical treatment. Such a diagnostic tool allows for time saving measures and improvement of the overall patient outcome.

In a further embodiment, the present invention provides a method of treating CCK-2R positive cancer in a subject in need thereof comprising administering to the subject an effective amount of compound according to the invention, or a pharmaceutically acceptable salt thereof.

In another embodiment, the present invention provides a compound according to the invention, or a pharmaceutically acceptable salt thereof, for use in treating CCK-2R positive cancer in a subject in need thereof.

In yet a further embodiment, the invention provides use of a compound according to the invention, or a pharmaceutically acceptable salt thereof, in the manufacture of a medicament for diagnosing and/or treating a CCK-2R positive cancer in a subject in need thereof.

Typically, radionuclides suitable for the treatment of cancer are beta-emitting radionuclides. After administration and localisation of the compounds to CCK-2R positive cancer the radionuclide complexed to the compound of the invention directly interacts with the tumour cell. Radionuclides for treating cancer that are within the scope of the invention include, but are not limited to, copper-67, yttrium-90, iodine-131, terbium-149, terbium-161, lutetium-177, rhenium-186, rhenium-188, bismuth-212, bismuth-213, astatine-211, lead-212 and actinium-225.

As used herein, the term “subject” refers to an animal, such as a bird or a mammal. Specific animals include rat, mouse, dog, cat, cow, sheep, horse, pig or primate. A subject may be a human, alternatively referred to as a patient. A subject may further be a rodent, such as a mouse or a rat.

In general, techniques for preparing the compounds of the invention are well known in the art, for example, see:
a) Alewood, P.; Alewood, D.; Miranda, L.; Love, S.; Meutermans, W.; Wilson, D. Meth. Enzymol., 1997, 289, 14-28;
b) Merrifield, R. B., J. Am. Chem. Soc., 1964, 85, 2149;
c) Bodanzsky, “Principles of Peptide Synthesis”, 2nd Ed., Springer-Verlag (1993); and
d) Houghten, Proc. Natl. Acad. Sci. USA, 1985, 82, 5131.

Known solid or solution phase techniques may be used in the synthesis of the compounds of the present invention, such as coupling of the N- or C-terminus to a solid support (typically a resin) followed by step-wise synthesis of the linear peptide. Protecting group chemistries for the protection of amino acid residues, including side chains, are well known in the art and may be found, for example, in: Theodora W. Greene and Peter G. M. Wuts, Protecting Groups in Organic Synthesis (Third Edition, John Wiley & Sons, Inc, 1999), the entire contents of which is incorporated herein by reference.

Briefly, the compounds of the invention were synthesised according to general scheme 1 below.

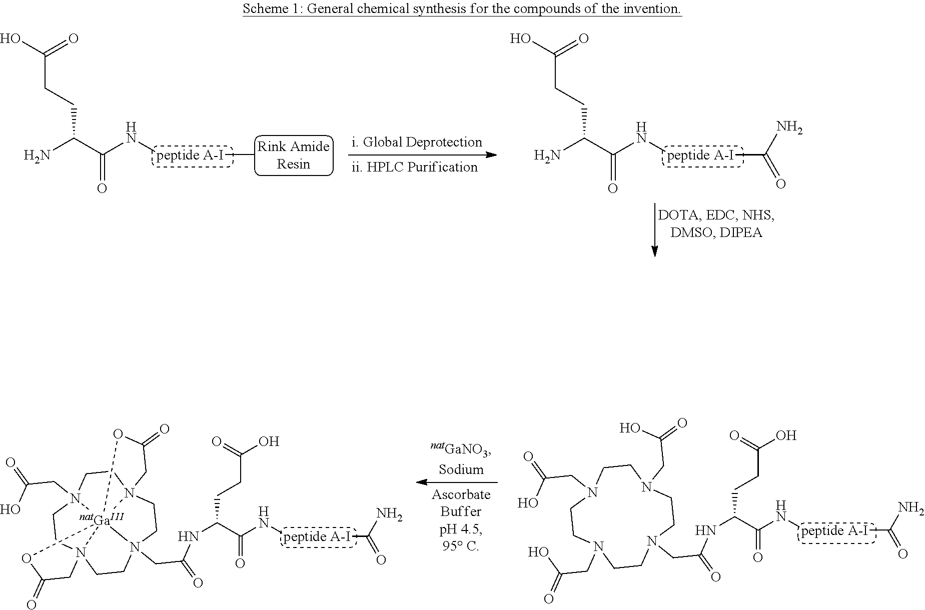

N-Terminus free linear peptides A to I were synthesised using general Fmoc-based solid phase peptide (SPPS) synthesis protocols using microwave assisted automated CEM Liberty peptide-synthesis module. The N-terminus free linear peptides A-I were globally deprotected and cleaved off the resin, HPLC purified and then conjugated to the radionuclide binding ligand (illustrated as DOTA) in solution. The compounds were then coordinated to a radionuclide or to natural gallium for binding assays in sodium ascorbate buffered solution (pH 4.5) to afford the coordinated compounds in quantitative yields.

Where the compounds of the present invention require purification, chromatographic techniques such as reversed-phase high-performance liquid chromatography (HPLC) may be used. The compounds may be characterised by mass spectrometry and/or other appropriate methods.

Where the compound comprises one or more functional groups that may be protonated or deprotonated (for example at physiological pH) the compound may be prepared and/or isolated as a pharmaceutically acceptable salt. It will be appreciated that the compound may be zwitterionic at a given pH. As used herein the expression “pharmaceutically acceptable salt” refers to the salt of a given compound, wherein the salt is suitable for administration as a pharmaceutical. Such salts may be formed, for example, by the reaction of an acid or a base with an amine or a carboxylic acid group, respectively. In the case of a zwitterionic compound, the salt may be an internal salt where the compound comprises suitable proton donating and accepting functional groups.

Pharmaceutically acceptable acid addition salts may be prepared from inorganic and organic acids. Examples of inorganic acids include hydrochloric acid, hydrobromic acid, sulfuric acid, nitric acid, phosphoric acid and the like. Examples of organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

Pharmaceutically acceptable base addition salts may be prepared from inorganic and organic bases. Corresponding counter ions derived from inorganic bases include the sodium, potassium, lithium, ammonium, calcium and magnesium salts. Organic bases include primary, secondary and tertiary amines, substituted amines including naturally-occurring substituted amines, and cyclic amines, including isopropylamine, trimethyl amine, diethylamine, triethylamine, tripropylamine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, and N-ethylpiperidine.

Acid/base addition salts tend to be more soluble in aqueous solvents than the corresponding free acid/base forms.

The compounds of the invention may be in crystalline form or as solvates (e.g. hydrates) and it is intended that both forms are within the scope of the present invention. The term “solvate” is a complex of variable stoichiometry formed by a solute (in this invention, a peptide of the invention) and a solvent. Such solvents should not interfere with the biological activity of the solute. Solvents may be, by way of example, water, ethanol or acetic acid.

Methods of solvation are generally known within the art.

The present invention also provides a pharmaceutical composition comprising the compound according to the invention, together with at least one pharmaceutically acceptable carrier or diluent.

As will be readily appreciated by those skilled in the art, the route of administration and the nature of the pharmaceutically acceptable carrier will depend on the nature of the condition and the mammal to be treated. Radiolabelled compounds are generally administered to a subject, intravenously, enterally or parenterally, as therapeutic and/or diagnostic agents. It is believed that the choice of a particular carrier or delivery system, and route of administration could be readily determined by a person skilled in the art. In the preparation of any formulation containing the compound according to the invention care should be taken to ensure that the activity of the compound is not destroyed in the process and that the compound is able to reach its site of action without being destroyed. Similarly the route of administration chosen should be such that the compound reaches its site of action.

Those skilled in the art may readily determine appropriate formulations for the compounds of the present invention using conventional approaches. Identification of preferred pH ranges and suitable excipients, for example antioxidants, is routine in the art. Buffer systems are routinely used to provide pH values of a desired range and include carboxylic acid buffers for example acetate, citrate, lactate and succinate. A variety of antioxidants are available for such formulations including phenolic compounds such as BHT or vitamin E, reducing agents such as methionine or sulphite, and metal chelators such as EDTA.

It is envisaged that the compounds according to the invention will be prepared in parenteral dosage forms, including those suitable for intravenous, intrathecal, and intracerebral or epidural delivery. The pharmaceutical forms suitable for injectable use include sterile injectable solutions or dispersions, and sterile powders for the extemporaneous preparation of sterile injectable solutions. They should be stable under the conditions of manufacture and storage and may be preserved against reduction or oxidation and the contaminating action of microorganisms such as bacteria or fungi.

The solvent or dispersion medium for the injectable solution or dispersion may contain any of the conventional solvent or carrier systems for the active compound, and may contain, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about where necessary by the inclusion of various antibacterial and antifungal agents, for example, parabens, chlorobutanol, phenol, sorbic acid, thimerosal and the like. In many cases, it will be preferable to include agents to adjust osmolarity, for example, sugars or sodium chloride. Preferably, the formulation for injection will be isotonic with blood. Prolonged absorption of the injectable compositions can be brought about by the use in the compositions of agents delaying absorption, for example, aluminium monostearate and gelatin. Pharmaceutical forms suitable for injectable use may be delivered by any appropriate route including intravenous, intramuscular, intracerebral, intrathecal, epidural injection or infusion.

Sterile injectable solutions are prepared by incorporating the aqueous liquids of the invention in the required amount in the appropriate solvent with various of the other ingredients such as those enumerated above, as required, followed by filtered sterilization. Generally, dispersions are prepared by incorporating the various sterilised active ingredient into a sterile vehicle which contains the basic dispersion medium and the required other ingredients from those enumerated above.

Pharmaceutically acceptable vehicles and/or diluents include any and all solvents, dispersion media, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate the compositions in unit dosage form for ease of administration and uniformity of dosage. Unit dosage form as used herein refers to physically discrete units suited as unitary dosages for the mammalian subjects to be diagnosed; each unit containing a predetermined quantity of the nanoparticle calculated to produce the desired diagnosis in association with the required pharmaceutically acceptable vehicle. The specification for the novel unit dosage forms of the invention are dictated by and directly dependent on (a) the unique characteristics of the nanoparticle and the particular diagnosis to be achieved, and (b) the limitations inherent in the art of compounding the nanoparticles on the invention in living subjects having a diseased condition in which bodily health is impaired.

As mentioned above, the principal active ingredient may be compounded for convenient and effective administration in effective amounts with a suitable pharmaceutically acceptable vehicle in unit dosage form. A unit dosage form can, for example, contain the nanoparticles in amounts ranging from 0.25 μg to about 2000 mg. Expressed in proportions, the active compound may be present in from about 0.25 μg to about 2000 mg/mL of carrier. In the case of compositions containing supplementary active ingredients, the dosages are determined by reference to the usual dose and manner of administration of the said ingredients.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The invention will now be described with reference to the following non-limiting examples:

### Example 1. Investigation of D-Alanine Insertion in the Hexa-Glutamic Acid Sequence

A series of peptides were synthesised in which one of the glutamic acid residues in the hexa-glutamic acid sequence of CP04, for example, was replaced with a D-glutamic acid residue. In addition, the Gly-Trp-Met-Asp-Phe-NH₂ (SEQ ID NO: 28) sequence of CP04 was replaced with NMe-Gly-Trp-NMe-Nle-Asp-Nal. The alanine-substituted peptides were then evaluated for their ability to disrupt the binding between CCK-2 receptor (CCK-2R) and [¹⁷⁷Lu]Lu-DOTA-CP04.

### 1.1. Peptide Synthesis

Peptides were assembled using standard Fmoc-based solid phase peptide synthesis (SPPS) procedures using Rink amide resin (0.8 mmol/g) on an automated CEM Liberty Blue microwave peptide synthesizer (John Morris Group, Victoria, Australia). Peptides were assembled on a 0.1 mmol reaction scale. Fmoc-deprotection was performed in two stages as following. The peptide-resin was treated with 20% piperidine/DMF (v/v; 5 mL) containing oxyma (0.1 M) under microwave irradiation for 30 s (40 W, 40° C.). This was followed by filtration and a second treatment of the same deprotection cocktail under microwave irradiation (45 W, 75° C.; 3 min). The peptide-resins were then rinsed with DMF (3×4 mL). Coupling of all standard Fmoc-amino acids was achieved by the addition of Fmoc-amino acid (5 eq, 0.5 mmol), DIC (5 eq) and oxyma (10 eq) in DMF (4 mL) to the Nu-deprotected peptide-resin and the mixture agitated under microwave radiation for 3 min (30 W, 90° C.). Following sequence assembly, the peptide-resins were rinsed manually with dichloromethane (DCM) (3×5 mL) prior to the cleavage step. Global deprotection and cleavage of peptides from the solid support was performed using TFA/TIPS/H2O:DODT:1,3-dimethoxybenzene (85:5:5:2.5:2.5, % v/v/v/v/v; 8 mL) cocktail for 2 h. The cleavage mixtures were then filtered, the TFA solutions evaporated under nitrogen flow and the crude products isolated by trituration and subsequent washes with diethyl ether (3×30 mL).

Peptides were purified using a Aeris 5 um PEPTIDE XB-C18 column (10 μm, 100 Å, 250×21.2 mm) eluted at 8 mL/min with a gradient of MeCN: 0.1% (v/v) TFA.

### 1.2. Radiosynthesis of [¹⁷⁷Lu]Lu-DOTA-CP04 for In Vitro Ligand Binding Assays

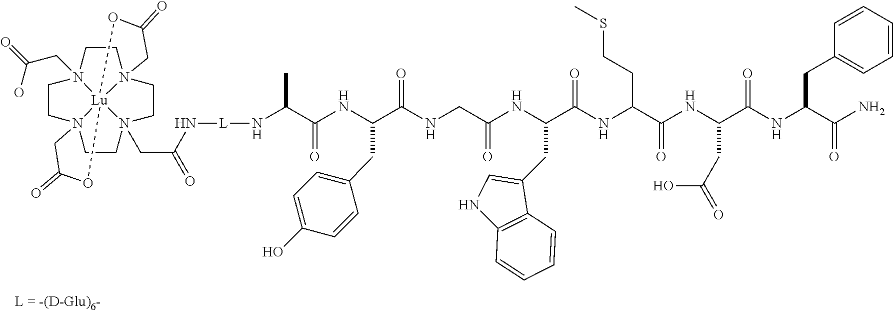

[¹⁷⁷Lu]Lu-DOTA-CP04 was prepared similarly to a reported method with slight variations (Ocak, M. et al., Eur J Nucl Med Mol Imaging 2011, 38(8), 1426-35). Briefly, DOTA-CP04 (30 μg, 14.6 nmol) dissolved in sodium acetate (0.5M, 100 μL (0.3 μg/μL peptide solution)) was constituted in 0.4 M ammonium acetate/0.24 M 2,5-dihydroxybenzoic acid (200 μL, pH 4.5) containing ethanol (50 μL), L-methionine (50 μL of 10 mg/mL solution in milliQ water) and sodium ascorbate (50 μL, 0.05M in milliQ water). 500-1000 MBq of non-carrier added [¹⁷⁷Lu]LuCl₃ in 0.04M HCl (50-100 μL) was added to this mixture and the resulting solution was heated at 80° C. for 20 min. A fraction of this material containing ca. 200 MBq was HPLC purified using a Proteo 90 Å (250×4.6 mm) eluted at 1 mL/min with a gradient of MeCN containing 0.1% TFA and 2% EtOH (v/v), starting at 25% MeCN for 1 min, increased to 65% over 80 min. Product eluting at 35 min was manually collected in a vial containing water:ethanol (9:1 v/v, 10 ml) and then trapped on a Strata-X SPE cartridge. The product was then eluted of the cartridge using ethanol (1 mL). A fraction of this purified peptide was then diluted in Dulbecco's Modified Eagle's medium (DMEM) supplemented with 1% foetal calf serum to afford 4 MBq/mL final concentration suitable for ligand binding assays.

### 1.3 Ligand Binding Assays

CCK-2 receptor binding experiments were conducted using A431 (human epidermoid carcinoma) cells stably transfected to over-express the human full-length CCK-2 receptor (A431-CCK-2R). As a negative control, A431 cells stably transfected with an empty vector were analysed simultaneously (A431-EV). A431 cells were maintained in DMEM (Gibco, Australia) media supplemented with 10% foetal calf serum and 250 μg/mL G418 as described previously (Aloj L, et al., J Nucl Med., 2004, Mar; 45(3), 485-94). The affinity of peptides for the CCK-2R was evaluated using competitive binding assays against [¹⁷⁷Lu]Lu-DOTA-CP04 in A431-CCK-2R cells. Briefly, 48 h before the experiment cells were plated at a density of 650,000 cells per well in 6 well plates. On the day of the experiment the peptides were diluted in DMEM supplemented with 1% foetal calf serum to at least 7 different concentrations (0-400 nM) and approximately 50,000 cpm of [¹⁷⁷Lu]Lu-DOTA-CP04 was added to each dilution. Cells were washed twice in ice-cold binding buffer (DMEM supplemented with 1% foetal calf serum) and resuspended in 1.5 mL of the pre-prepared compound mix before being incubated at 37° C. for 1 h. Post-incubation the media was removed and kept for analysis (unbound fraction) and the cells were washed twice in ice-cold binding buffer, which was pooled with the unbound fraction. Finally, 1 M NaOH was added to the cells and allowed to incubate for 5 min before lysates were collected (bound fraction). A small volume of lysate was kept aside to determine protein concentration using the Pierce® BCA protein assay kit (Thermo Fisher, Australia). All samples were analysed using the Perkin Elmer 2480 Wizard2™ gamma counter (PerkinElmer, Massachusetts, USA) and were normalised according to protein concentration. Half-maximal inhibitory concentrations (IC₅₀) were determined using nonlinear regression calculated using GraphPad Prism Software (GraphPad Software, California, USA).

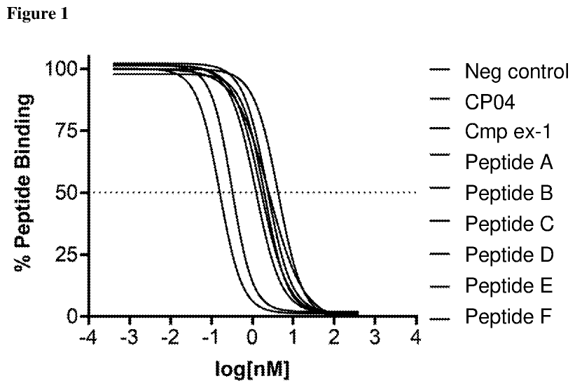

The apparent binding affinities (IC₅₀) (Table 2; FIG. 1) of the D-Ala substituted peptides compared to comparative example-1 and CP04 demonstrate the impact of D-Ala substitution on each of the glutamic acid residues on the critical hexameric peptide sequence. CP04, when tested against [¹⁷⁷Lu]Lu-DOTA-CP04, showed greater than 3-fold decrease in affinity for the CCK-2 receptor, from 0.76 nM to 2.33 nM. This loss of affinity very clearly demonstrates the importance of the linker and the radio-labelled complex in improving affinity of the peptide to the receptor of interest. Similarly, comparative example-1, comprising the hexa-glutamic acid sequence, had previously been assessed in its ^{nat}Ga DOTA-peptide form with an affinity of 0.05 nM for CCK-2 receptor, but observed a 5-fold reduction in its affinity to the receptor. Despite this, comparative example-1 established an important baseline for the D-Ala analogues, with an IC₅₀ of 0.32 nM.

TABLE 2
Relative binding affinity (IC₅₀) for D-Ala substituted peptides

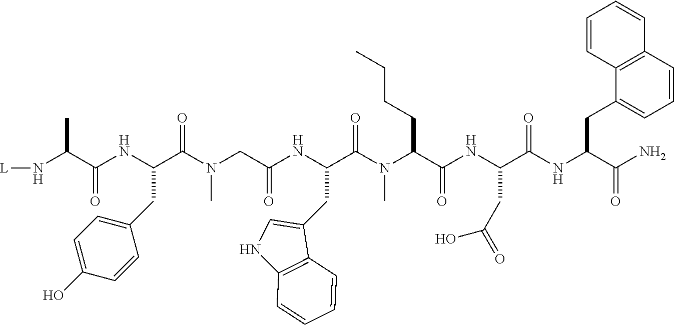

| Peptide | -L- | IC₅₀ (nM) | SEQ ID NO: |
|---|---|---|---|
| A | -D-Ala-D-Glu-D-Glu-D-Glu-D-Glu-D- | 3.071 | 16 |
| | Glu-(SEQ ID NO: 30) | | |
| B | -D-Glu-D-Ala-D-Glu-D-Glu-D-Glu-D- | 1.233 | 17 |
| | Glu-(SEQ ID NO: 31) | | |
| C | -D-Glu-D-Glu-D-Ala-D-Glu-D-Glu-D- | 0.204 | 1 |
| | Glu-(SEQ ID NO: 32) | | |
| D | -D-Glu-D-Glu-D-Glu-D-Ala-D-Glu-D- | 1.618 | 18 |
| | Glu-(SEQ ID NO: 33) | | |
| E | -D-Glu-D-Glu-D-Glu-D-Glu-D-Ala-D- | 2.822 | 19 |
| | Glu-(SEQ ID NO: 34) | | |
| F | -D-Glu-D-Glu-D-Glu-D-Glu-D-Glu-D- | 1.617 | 20 |
| | Ala-(SEQ ID NO: 35) | | |
| Cmp ex-1 | -(D-Glu)₆-(SEQ ID NO: 36) | 0.319 | 21 |
| CP04 | | 2.325 | |

Peptide A, with D-alanine substituted at terminal position 1, saw a significant loss of binding affinity to the receptor of interest. Substitution with the small D-alanine residue at position 1 clearly demonstrates the important role of the terminal glutamic acid in improving the binding affinity of the peptide sequence to the CCK-2 receptor, as observed by the greater than 10-fold loss of activity. Similarly, peptide E with D-alanine substituted at position 5 observed an equivalent loss of activity for the receptor. Previous work in this scaffold has shown the peptide preferentially folds into a β-hairpin structure through backbone and residue intramolecular interactions and this secondary structure is critical for its exquisite activity. The D-alanine substitutions would suggest that the D-glutamic acid residues at positions 1 and 5 are critical in the formation of this secondary structure and therefore loss of these critical residues results in loss of activity.

Evaluating the biological activity of Peptides B, D and F, which had iterative D-alanine substitution in positions 2, 4, 6, it was found that the removal of the D-glutamic acid moiety was well tolerated whilst maintaining moderate affinity to the CCK-2 receptor. The resulting activity exhibited an approx. 5-fold loss in binding affinity to target receptor across all three peptides. This result provides some insight into what other possible substitutions can be made without severely impairing the affinity between the ligand and receptor, unlocking numerous opportunities for future modifications.

Peptide C, with substitution of D-alanine at position 3, yielded the most active ligand of the library of peptides evaluated. With an IC₅₀ of 0.18 nM, Peptide C displayed a greater than 10-fold improvement upon the activity of the established CP04. Additionally, it exceeded the already outstanding affinity of the comparative peptide by almost 2-fold. Position 3 therefore is not only highly tolerable of modifications but substitution of a neutral hydrophobic moiety such as D-alanine greatly improves the binding between the ligand and receptor. Considering the 3-dimensional β-hairpin structure of this scaffold, it is very possible that the highly polar and bulky D-glutamic acid at this position repels the corresponding residue on the binding site of CCK-2 receptor while the small, neutral, hydrophobic D-alanine residue is far more suitable to the binding pocket of the receptor.

A further study was conducted to assess the binding affinity of peptides based on Cmp ex-1 in which the 3-(1-naphthyl)alanine residue was replaced with various substituted-phenylalanine residues or with 3-(2-naphthyl)-L-alanine (Table 3). Most of resultant peptides showed comparable affinity to Cmp ex-1 or CP04. Notably, substitution of 3-(1-naphthyl)alanine with 3,4,5-trifluoro-phenylalanine resulted in increased affinity over the comparative examples.

TABLE 3
Relative binding affinity (IC₅₀) for substituted-phenylalanine peptides

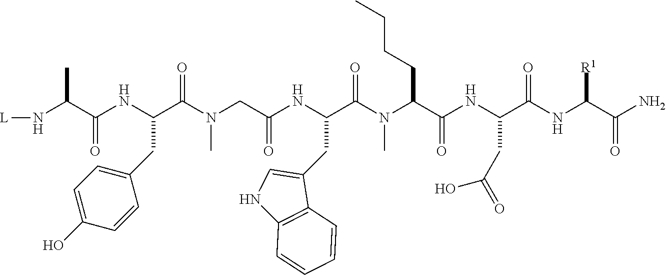

| Peptide | -L- | R¹ | IC₅₀ (nM) | SEQ ID NO: |
|---|---|---|---|---|
| G | -(D-Glu)₆- | 4-fluoro-L-Phe | 0.92 | 22 |
| H | -(D-Glu)₆- | 3,4-difluoro-L-Phe | 0.64 | 23 |
| I | -(D-Glu)₆- | 3,4,5-trifluoro-L-Phe | 0.26 | 24 |
| J | -(D-Glu)₆- | 2,3,4,5,6-pentafluoro-L-Phe | 1.92 | 25 |
| K | -(D-Glu)₆- | 4-amino-L-Phe | 0.52 | 26 |
| L | -(D-Glu)₆- | 3-(2-naphthyl)-L-alanine | 0.60 | 27 |
| Cmp ex-1 | -(D-Glu)₆- | 3-(1-naphthyl)-L-alanine | 0.33 | 21 |
| CP04 | | | 3.17 | |

Finally, a study was conducted to assess the binding affinity of peptides based on Peptide C, with substitution of D-alanine at position 3, in which the 3-(1-naphthyl)alanine residue was replaced with various substituted-phenylalanine residues, with tyrosine or with 3-(2-naphthyl)-L-alanine (Table 4). Most of resultant peptides showed comparable affinity to Peptide C.

TABLE 4
Relative binding affinity (IC₅₀) for modified peptides based on Peptide C

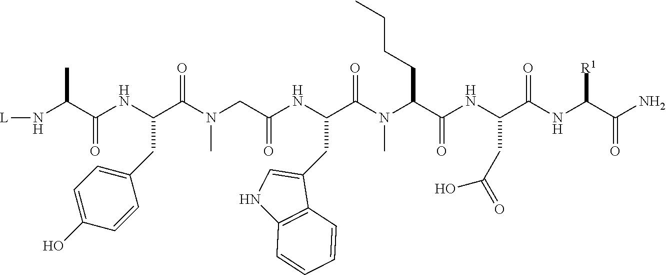

| Peptide | -L- | R¹ | IC₅₀ (nM) | SEQ ID NO: |
|---|---|---|---|---|
| C | -(D-Glu)₂-D-Ala-(D- | 3-(1-naphthyl)-L- | 0.204 ± 0.015 | 1 |
| | Glu)₃- | alanine | | |
| M | -(D-Glu)₂-D-Ala-(D- | 4-amino-L-Phe | 0.741 ± 0.064 | 8 |
| | Glu)₃- | | | |
| N | -(D-Glu)₂-D-Ala-(D- | 3,4,5-trifluoro-L-Phe | 0.444 ± 0.032 | 7 |
| | Glu)₃- | | | |
| O | -(D-Glu)₂-D-Ala-(D- | 4-fluoro-L-Phe | 0.368 ± 0.040 | 5 |
| | Glu)₃- | | | |
| P | -(D-Glu)₂-D-Ala-(D- | 4-trifluoromethyl-L-Phe | 1.143 | 11 |
| | Glu)₃- | | | |
| Q | -(D-Glu)₂-D-Ala-(D- | 4-cyano-L-Phe | 1.568 ± 0.089 | 10 |
| | Glu)₃- | | | |
| R | -(D-Glu)₂-D-Ala-(D- | Tyr | 1.146 ± 0.096 | 12 |
| | Glu)₃- | | | |
| S | -(D-Glu)₂-D-Ala-(D- | 3,4-difluoro-L-Phe | 2.657 ± 0.226 | 6 |
| | Glu)₃- | | | |
| T | -(D-Glu)₂-D-Ala-(D- | 2,3,4,5,6-pentafluoro-L- | 0.607 ± 0.052 | 13 |
| | Glu)₃- | Phe | | |
| U | -(D-Glu)₂-D-Ala-(D- | 3-fluoro-L-Phe | 0.305 ± 0.013 | 14 |
| | Glu)₃- | | | |
| V | -(D-Glu)₂-D-Ala-(D- | 4-acetyl-L-Phe | 0.948 ± 0.052 | 15 |
| | Glu)₃- | | | |

### Example 2. Investigation of C-Terminal Modification of Peptide C

Following on from the D-alanine study, a series of compounds were synthesised in which the NMe-Gly-Trp-NMe-Nle-Asp-Nal sequence of Peptide C was modified. The radionuclide binding ligand DOTA was then conjugated to the peptide and chelated with ^{nat}Gallium

### 2.1. Synthesis and Purification of DOTA-Conjugated Compounds

Synthesis and purification of the peptides was performed as described above. The conjugation of free DOTA to the peptides was conducted in solution analogously to a previously reported procedure with few modifications (Schottelius, M.; Schwaiger, M.; Wester, H. J., Tetrahedron Letters, 2003, 44(11), 2393-2396). Briefly, DOTA (1.5 eq. relative to peptide) was pre-activated with N-hydroxysuccinimide (NHS) (1.5 eq. relative to NHS) using EDCI (1.5 eq. relative to NHS) and DIPEA (2 eq. relative to NHS) in anhydrous DMSO (typically 500 μL). The reaction mixture was sonicated at 50° C. for 30 min until all starting material completely dissolved. The peptides were then added in a minimum volume of anhydrous DMSO (typically 200-500 μL). The resulting mixture is left shaking for a further 30 min at room temperature and the reaction progress was monitored by MS analysis. When reaction was complete indicated by the disappearance of the m z peak of the starting peptide in MS analysis, distilled water was added to form a 20:80 mixture of DMSO:water and the crude peptide was purified using reverse phase HPLC. HPLC conditions for the purification of DOTA-compounds were identical to those reported above for the purification of the respective compounds.

TABLE 4
Chemical structure of Compounds 1-4 and comparative example-2.

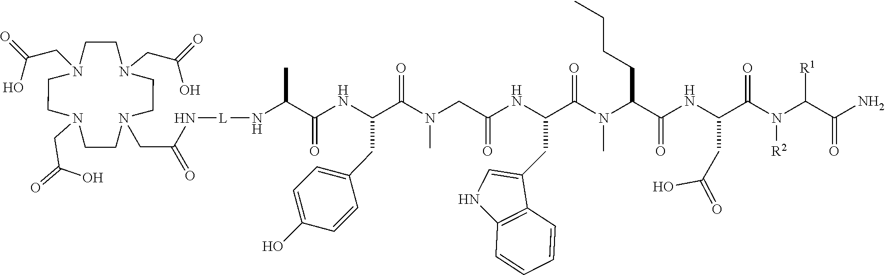

| Compound # | -L- | R¹ | R² |
|---|---|---|---|
| 1 | -(D-Glu)₂-D-Ala-(D-Glu)₃- | Nal | H |
| | (SEQ ID NO: 32) | | |
| 2 | -(D-Glu)₂-D-Ala-(D-Glu)₃- | Phe | methyl |
| | (SEQ ID NO: 32) | | |
| 3 | -(D-Glu)₂-D-Ala-(D-Glu)₃- | Phe | H |
| | (SEQ ID NO: 32) | | |
| 4 | -(D-Glu)₂-D-Ala-(D-Glu)₃- | Nal | methyl |
| | (SEQ ID NO: 32) | | |
| Cmp-ex 02 | -(D-Glu)₆- (SEQ ID NO: 29) | Nal | methyl |

2.2. Synthesis and purification of ^{nat}Gallium chelated DOTA-compounds

The chelation of ^{nat}Gallium to DOTA-compounds was performed in accordance with a published procedure (Maurin, M., et al., Nucl Med Rev Cent East Eur., 2015, 18(2), 51-5). Briefly, the compounds (2-8 μmol) were suspended in a freshly prepared ascorbic acid buffer (3-5 mL of 50 mg/mL) neutralised with a concentrated solution of NaOH to pH 4.5. Gallium nitrate (4 eq. relative to DOTA-peptide) was then added and the resulting mixture was heated at 95° C. for 15 min. The resulting mixture was cooled to room temperature, centrifuged to remove any precipitate and purified using reverse phase HPLC adopting the same chromatographic conditions for the purification of the respective N-terminus free peptides.

### 2.3. Receptor Binding Assays

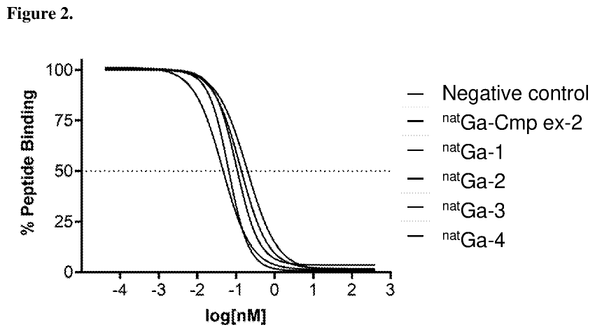

The compounds were evaluated for their ability to disrupt the binding between CCK-2 receptor (CCK-2R) and [¹⁷⁷Lu]Lu-DOTA-CP04 using the methodology described above and compared to comparative example-2 (FIG. 2; Table 5).

TABLE 5
Relative binding affinity (IC₅₀) for compounds based on Peptide C

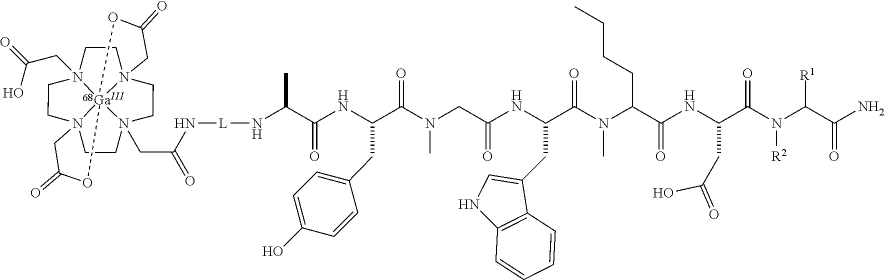

| Compound | -L- | R¹ | R² | IC₅₀ (nM) |
|---|---|---|---|---|
| ^{nat}Ga-1 | -(D-Glu)₂-D-Ala-(D-Glu)₃- | Nal | H | 0.080 |
| | (SEQ ID NO: 32) | | | |
| ^{nat}Ga-2 | -(D-Glu)₂-D-Ala-(D-Glu)₃- | Phe | methyl | 0.185 |
| | (SEQ ID NO: 32) | | | |
| ^{nat}Ga-3 | -(D-Glu)₂-D-Ala-(D-Glu)₃- | Phe | H | 0.042 |
| | (SEQ ID NO: 32) | | | |
| ^{nat}Ga-4 | -(D-Glu)₂-D-Ala-(D-Glu)₃- | Nal | methyl | 0.096 |
| | (SEQ ID NO: 32) | | | |
| ^{nat}Ga-Cmp ex-2 | -(D-Glu)₆- (SEQ ID NO: 29) | Nal | methyl | 0.158 |

Comparative example-2 (Cmp ex-2), previously evaluated in several preliminary studies, exhibited advantageous biostability, selectivity for target receptor and the highest tumour uptake in biodistribution mouse studies when evaluated alongside other potential ligand scaffolds. However, this peptide also unfortunately had the highest renal uptake of compounds tested. This is in large part due to the highly acidic hexameric terminal peptide chain, known to promote kidney uptake. Cmp ex-2 serves as an important template to investigate the impact of D-alanine substitution, reducing the overall acidic nature of the peptide, and exploring the impact of this structural change on binding affinity of the peptide to the receptor of interest. Compounds 1-4 were designed with changes to the critical C-terminal recognition sequence residues.

The importance of the (^{nat}Ga)-DOTA conjugated peptide in improving binding affinity of the linear peptide to receptor has previously been determined. So to had the hexa-D-glutamic acid substituted variants of Compounds 1, 3, and 4 been previously investigated and shown to have sub-nanomolar affinity to the CCK-2 receptor. High aromaticity in the C-terminal position promotes improved binding to the receptor. Further constraining the peptide by introducing N-methylation at key residues stabilises the peptide but does see a marginal decrease in affinity. To determine the impact of D-alanine substitution at position 3 of these peptides, the ligands were assayed and shown to tolerate the substitution extremely well, with minimal loss of binding affinity across all compounds tested.

### Example 3. Radiosynthesis of Gallium-68 Labelled DOTA-Compounds

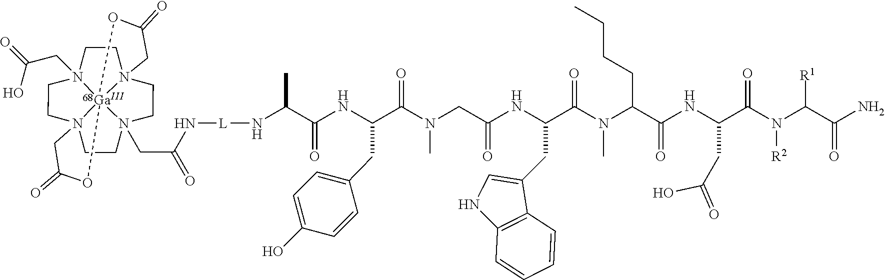

### 3.1. Radiolabelling DOTA-Compounds

DOTA-Compounds 1-4 and comparative example-2 were radiolabelled in accordance with a published procedure (Haskali, M. B. et al., EJNMMI Radiopharmacy and Chemistry, 2019, 4(1), 23). Briefly, a mixture of the corresponding DOTA-peptide precursor (30 μg) in 0.5M sodium acetate solution (800 μL), ethanol (200 μL), 0.05M sodium ascorbate (200 μL), 0.05M 2,5-dihydroxybenzoic acid sodium salt (200 μL) and 10 mg/ml methionine (100 μL) was freshly prepared before radiosynthesis. The reaction mixture was then transferred into the reactor of a MultiSyn radiochemistry module. Gallium-68 was then delivered to the reaction vessel by elution of a ITG ⁶⁸Ge/⁶⁸Ga generator using 0.05M HCl (4 mL). The reaction mixture was heated to 90° C. for 480 s (pH of reaction mixture is 4.5). The reaction mixture was then diluted with water (5 mL) and the gallium-68 labelled peptide was trapped on a Strata-X SPE cartridge. The trapped product was rinsed with water (5 mL), eluted with ethanol (˜0.5 mL) and diluted with saline (9 mL) to afford Compounds [⁶⁸Ga]Ga-1-4 and [⁶⁸Ga]Ga-Cmp ex-2 in in high radiochemical purity.

### 3.2. Quality Control of Gallium-68 Labelled DOTA-Compounds

Final formulation appearance, pH, radionuclidic identity (half-life test), purity and radiochemical identity were assessed for each of the radiolabelled compounds (Table 6). Radiochemical identity and purity was assessed by radio-HPLC analysis of Compounds [⁶⁸Ga]Ga-1-4 and [⁶⁸Ga]Ga-Cmp ex-2. Radiochemical identity was confirmed by matching retention time (and co-mobility) of the gallium-68 labelled Compounds and their respective non-radioactive reference standards (chelated with ^{nat}gallium). The radiochemical purity is identified by integration of all observed radioactive peaks and comparison of their relative % area.

TABLE 6
Quality control analysis of Compounds [⁶⁸Ga]Ga-1-4 and [⁶⁸Ga]Ga-Cmp ex-2. Specific
activity of radiopharmaceuticals used in our experiments varied depending on the amount
of Ga-68 eluted from the ITG 68Ge/68Ga generator, which is contingent on its age.

| | Parameter | | | | | |
|---|---|---|---|---|---|---|
| | | | | | Radiochemical | |
| | | | Radionuclidic | Radiochemical | purity (TLC) | Specific |
| | Appearance | | identity | purity (HPLC) | (≥90% labelled | activity |
| | (clear and | pH | (half-life) | (≥90% labelled | peptide; ≤2% | (≥3.0 |
| Compound # | colourless) | (4-8) | (62-74 min) | peptide) | free Ga-68) | MBq/μg) |
| [⁶⁸Ga]Ga-Cmp | Pass | 5-6 | Pass | 95-98% | >98% | 7-10 |
| ex-2 | | | | | <2% | MBq/μg |
| [⁶⁸Ga]Ga-1 | Pass | 5-6 | Pass | 92-96% | >98% | 7-10 |
| | | | | | <2% | MBq/μg |
| [⁶⁸Ga]Ga-2 | Pass | 5-6 | Pass | 93-96% | >98% | 7-10 |
| | | | | | <2% | MBq/μg |
| [⁶⁸Ga]Ga-3 | Pass | 5-6 | Pass | 92-94% | >98% | 7-10 |
| | | | | | <2% | MBq/μg |
| [⁶⁸Ga]Ga-4 | Pass | 5-6 | Pass | 93-95% | >98% | 7-10 |
| | | | | | <2% | MBq/μg |

### Example 4. LogD_{7.4} Determination and Serum Protein Binding Assays

The distribution coefficients (LogD_{7.4}) of radiolabelled compounds [⁶⁸Ga]Ga-1-4, and [⁶⁸Ga]Ga-Cmp ex-2 were assayed by measuring radioactivity distribution in the aqueous phase (phosphate buffer pH7.4) and the organic phase (n-octanol). To determine the distribution coefficient (LogD), Ga-68 radiolabelled compounds were diluted to 20 pmol/mL in PBS (pH 7.4) and an equal volume of n-octanol was added. The mixture was vortexed vigorously for 10 cycles of 1 min at room temperature before being centrifuged for 6 min to separate the two phases. Equal volumes from each phase were aliquoted into individual counting tubes and analysed for 1 min using the Perkin Elmer 2480 Wizard2™ gamma counter (PerkinElmer, Massachusetts, USA). The distribution coefficient was calculated using the following equation

$$Log {D}_{oct / wat} = log ( \frac{[ cpm ] oct}{[ cpm ] PBS} ) .$$

Ga-68 labelled compounds [⁶⁸Ga]Ga-1-4 and [⁶⁸Ga]Ga-Cmp ex-2 displayed high hydrophilicity with low LogD_{7.4} values ranging from −4.1 to −2.5 (Table 7). These low LogD_{7.4} are consistent with other peptide based theranostics successfully employed in clinical studies leading to rapid renal excretion and low background uptake. Furthermore, metabolic stability and bioavailability is, in part, modulated by serum protein binding, which reduces glomerular filtration and enzymatic degradation and increases retention in tumours degradation (Smith, D., Di, L. & Kerns, E., Nat Rev Drug Discov. 2010, 9, 929-939). Compounds [⁶⁸Ga]Ga-1-4 demonstrated moderate to high degree of plasma protein binding (59-84%).

TABLE 7
LogD_{7.4} and protein binding (%) of [⁶⁸Ga]Ga-1-4 and
[⁶⁸Ga]Ga-Cmp ex-2. LogD_{7.4} values (n = 5) obtained after
incubation in PBS/Octanol mix. Serum protein binding results
after peptides were incubated with pooled human serum for 1 h at 37° C.

| | LogD_{7.4} | Plasma Protein Binding (%) |
|---|---|---|
| [⁶⁸Ga]Ga-Cmp ex-2 | −2.97 ± 0.13 | 82.43 ± 0.38 |
| [⁶⁸Ga]Ga-1 | −2.88 ± 0.09 | 78.69 ± 2.11 |
| [⁶⁸Ga]Ga-2 | −3.56 ± 0.01 | 59.75 ± 1.04 |
| [⁶⁸Ga]Ga-3 | −4.06 ± 0.14 | 63.62 ± 0.45 |
| [⁶⁸Ga]Ga-4 | −2.59 ± 0.02 | 83.16 ± 0.13 |

### Example 5. Circular Dichroism

Circular dichroism (CD) measurements were acquired on a Chirascan-plus spectropolarimeter (Applied photophysics, United Kingdom). The samples were prepared from the diluted NMR samples where applicable. The CD experiments were performed between 195 nm and 260 nm in triplicate with 1 nm step size, 1 nm bandwidth, 1 s time-per-point and 1 mm quartz cell (Starna, United Kingdom). Signal was recorded as millidegree and zeroed at 260 nm and normalized to give units of mean-residue ellipticity (MRE) according to the following equation:

$$MRE = \frac{θ}{C \times l \times N}$$

where θ is the recorded ellipticity in milli-degrees, C is the peptide concentration in dmol/L, 1 is the cell path-length in cm, and N is the number of residues per peptide.

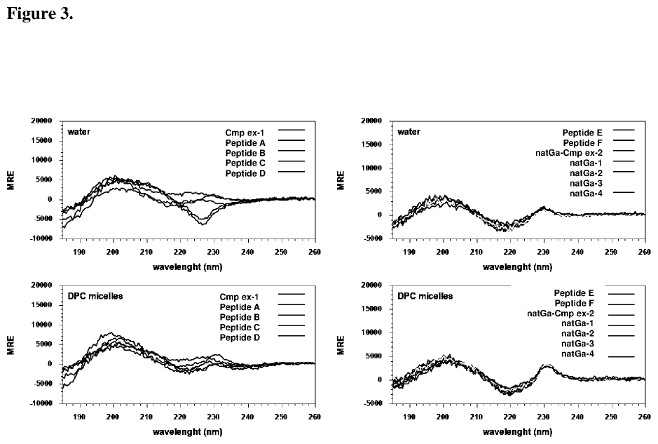

Peptides A-F, Compounds 1-4 and comparative examples 1 and 2 were analysed by CD spectroscopy in order to investigate any changes in secondary structure (FIG. 3). Peptides C and D exhibited a minima at ca. 228 nm in water, indicating an alpha-helical structure, which was resolved in DPC micelles. The overall line shape of comparative example-1 and Peptides A and B in water indicate random coil structures.

CD spectra of the remaining peptides and compounds exhibited two maxima at 200 nm and 230 nm and a minimum at ca. 220-227 nm.

### Example 6. In Vitro Metabolic Assays

Drug detoxification, drug metabolism and metabolic stability are important determinants of the efficacy of theranostic candidates in clinical settings. To assess metabolic stability, radiolabelled Compounds [⁶⁸Ga]Ga-1-4 and comparative example-2 ([⁶⁸Ga]Ga-Cmp ex-2) were challenged by incubation in either human serum, mouse liver or kidney homogenates and HEPES buffer as a negative control. Duplicate samples (750-900 pmol/mL) were incubated at 37° C. with pooled human serum, 15% mouse kidney homogenate, 30% mouse liver homogenate or 20 mM HEPES, pH 7.3 (control). Human blood was collected and serum prepared using SSTII advance vacutainer tubes according to the manufacturer's instructions (Becton Dickinson, New Jersey, USA). Mouse kidney and liver homogenates were prepared by washing the tissues in ice-cold HEPES buffer, pH 7.3 and placing them in pre-chilled tubes containing HEPES buffer and 2.38 mm metal beads (Mo-Bio Laboratories, Hilden, Germany). Tissues were homogenised using the PowerLyzer24 homogeniser (Qiagen, Hilden, Germany) according to the manufacturer's instructions. Ga-68 radiolabelled peptides were incubated with the various lysates at 37° C. and samples were collected at 15, 30, 60 and 90 min. Reactions were terminated by addition of an equal volume of acetonitrile and centrifugation for 2 min at 15,000 rpm. Supernatants were diluted in water before being analysed by HPLC using a Kinetex C18 XB column (5 μm 4.6×150 mm), eluted at 1.5 mL/min of gradient 20-60% MeCN in water containing 0.1% formic acid over 7 min.

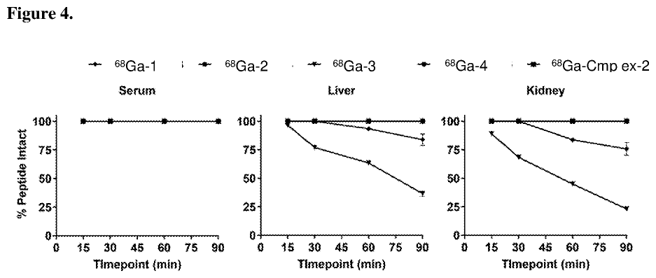

Experiments demonstrate that compounds tested remained intact when incubated with human serum at 37° C. over the 90 min time course (FIG. 4a). [⁶⁸Ga]Ga-1 and [⁶⁸Ga]Ga-3 both underwent significant metabolism in liver and kidney homogenates over the course of the experiment (FIG. 4b, 4c). [⁶⁸Ga]Ga-3 was particularly prone to degradation, with approximately 35% peptide intact in liver microsomes and only 20% peptide intact in kidney homogenates after 90 min. [⁶⁸Ga]Ga-1 showed a more stable metabolic profile, with 85% of peptide intact when incubated with liver microsomes and 75% of the parent peptide at 90 min. The remaining Compounds [⁶⁸Ga]Ga-2, [⁶⁸Ga]Ga-4, and [⁶⁸Ga]Ga Cmp ex-2 were extremely stable in liver and kidney homogenates, with no observable metabolites detected at the 90 min time point (FIG. 4).

Corlett and co-workers have previously demonstrated the metabolically labile nature of the terminal Phe residue that is present in Compound 3 (Corlett, A. et al., J. Med. Chem 2021, 64, 4841-4856). The results shown here remain consistent with those previously investigated, with Compound 3 exhibiting significant degradation at 90 min. Further analysis of the metabolised confirmed the cleavage site at position 13, the terminal Phe residue. Enzymatic cleavage at the Phe residue can be prevented by substituting the natural Phe moiety for the non-natural N-methylated Phe, as demonstrated in Compound 2. Incorporation of N-methylated residues is a known strategy to enhance protease resistance. This is demonstrated yet again in Compound 1 and its N-methylated variant at position 13, Compound 4. Although not as prone to metabolic degradation as Compound 3, N-methylation of the terminal naphthylalanine residue prevents enzymatic degradation of the peptide, leaving 100% of the parent peptide intact.

### Example 7. Small Animal PET Imaging and Biodistribution Studies

The diagnostic efficacy of Compounds [⁶⁸Ga]Ga-1-4 and [⁶⁸Ga]Ga-Cmp ex-2 was assessed using static small animal PET imaging and quantifying biodistribution studies. Female BALB/c nu/nu mice (age 8-10 weeks) were inoculated subcutaneously on the right flank with 3×10⁶ A431 CCK2R cells in PBS:Matrigel (1:1). Mice were weighed and tumours measured twice weekly using electronic callipers with tumour volume (mm³) calculated as length×width×height×π/6. Mice were assigned to imaging and/or biodistribution groups (tumour volumes: 50-500 mm³). The respective Ga-68 labelled tracer (4-5 MBq, 100 μL, 13.7-14.2 pmoles) was mixed with vehicle solution (75 μL, DMSO:Tween 80:water, 2:2:6 v/v/v) and then administered to six mice intravenously via tail vein injection. Three mice were euthanised for biodistribution 1 h post injection and selected tissues were excised, weighed and counted using a Capintec (Captus 4000e) gamma counter. Three mice were anaesthetised using 1.5% isoflurane and imaged at 1 and 2 h with a G8 Small Animal PET/CT scanner (Perkin Elmer/Sofie Biosciences). A 10 min static PET scan was acquired, followed immediately by a CT scan. PET images were acquired using the G8 acquisition engine software and reconstructed using a 3D maximal likelihood and expectation maximization (ML-EM) algorithm. PET images were analysed using VivoQuant software, version 3.0 (inviCRO Imaging Services and Software) to quantify maximum standardised uptake value in regions of interest (SUVmax). After imaging the 2 h time point the mice were harvested for biodistribution analysis as above.

For blocking CCK2R binding a solution of N-[(3R)-2,3-Dihydro-1-[2-(2-methylphenyl)-2-oxoethyl]-2-oxo-5-phenyl-1H-1,4-benzodiazepin-3-yl]-N′-(3-methylphenyl)-urea (YM022; Sigma Aldrich) was used. YM022 (75 μL of a 1 mg/mL solution constituting DMSO:Tween 80:water, 2:2:6 v/v/v) was added to the respective Ga-68 labelled tracer (4-5 MBq, 100 μL, 13.7-14.2 μmol) and this mixture (total 175 μL) was administered in three mice intravenously via tail vein injection and organs harvested at 1 h post injection for biodistribution analysis, as above. All animal studies were performed with the approval of the Peter MacCallum Cancer Centre Animal Experimentation Ethics Committee and in accordance with the Australian code for the care and use of animals for scientific purposes, 8th Edition, 2013.

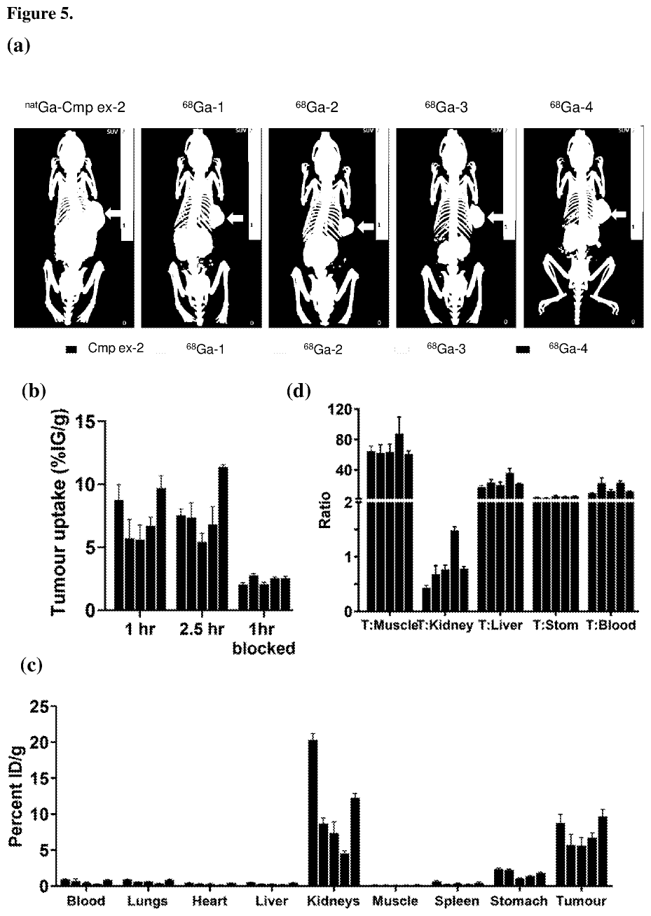

The imaging and biodistribution characteristics of Compounds [⁶⁸Ga]Ga-1-4 were compared to [⁶⁸Ga]Ga-Cmp ex-2 (FIG. 5). [⁶⁸Ga]Ga-Cmp ex-2 demonstrated the best tumour uptake. However, detrimentally for this compound, it also demonstrated the highest renal uptake. Compounds [⁶⁸Ga]Ga-1-4 had almost half, or less than half kidney uptake compared to [⁶⁸Ga]Ga-Cmp ex-2. Compound [⁶⁸Ga]Ga-4 had the best tumour to kidney ratio. All compounds tested had low biodistribution in blood, lungs, heart, liver and spleen.

1. A compound of Formula (I): 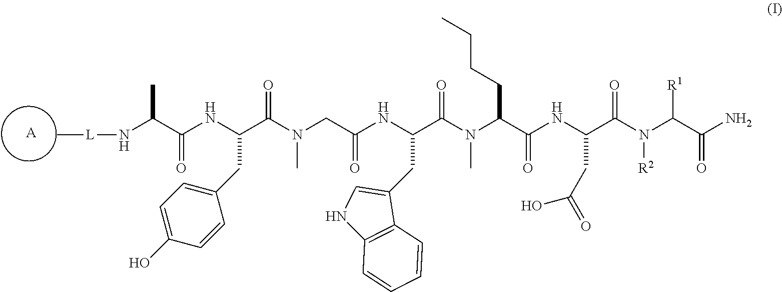
wherein
R¹ is a side chain of an amino acid selected from phenylalanine, 3-(1-naphthyl)alanine, 3-(2-naphthyl)alanine, or tyrosine, wherein the phenylalanine residue is optionally substituted with one or more halo, —NH₂, cyano, C₁₋₆alkyl, haloC₁₋₆alkyl, or acetyl;
R² is selected from H or C₁-C₃alkyl;
A is a radionuclide binding ligand; and
-L- is an amino acid sequence: -D-Glu-D-Glu-D-Ala-D-Glu-D-Glu-D-Glu- (SEQ ID NO: 32; or
a pharmaceutically acceptable salt thereof.

2. The compound, or the pharmaceutically acceptable salt thereof, according to claim 1 represented by the Formula (Ia): 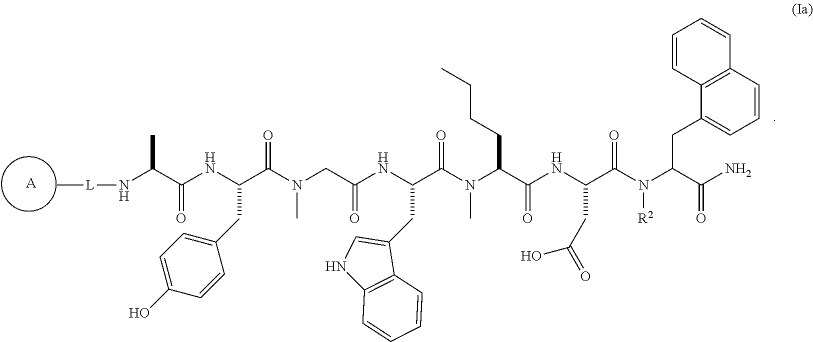
wherein R² is as defined in claim 1.

3. The compound, or the pharmaceutically acceptable salt thereof, according to claim 1 represented by the Formula (Ib): 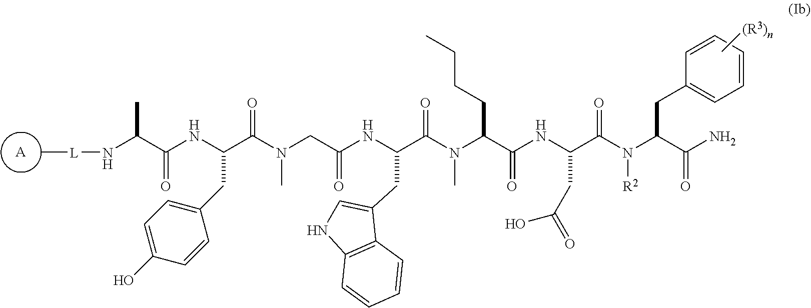
wherein R² is as defined in claim 1;
each occurrence of R³ is independently selected from halo, —NH₂, cyano, —OH, C₁₋₆alkyl, haloC₁₋₆alkyl, or acetyl; and
n is from 0 to 5.

4. The compound, or the pharmaceutically acceptable salt thereof, according to claim 3, wherein n is 0.

5. The compound, or the pharmaceutically acceptable salt thereof, according to claim 3, wherein R³ is F and n is from 1 to 3.

6. The compound, or the pharmaceutically acceptable salt thereof, according to claim 5, wherein n is 3.

7. The compound, or the pharmaceutically acceptable salt thereof, according to claim 5, wherein n is 1.

8. The compound, or the pharmaceutically acceptable salt thereof, according to claim 3, wherein R³ is —NH₂ and n is 1.

9. The compound, or the pharmaceutically acceptable salt thereof, according to claim 1 represented by the Formula (Ic): 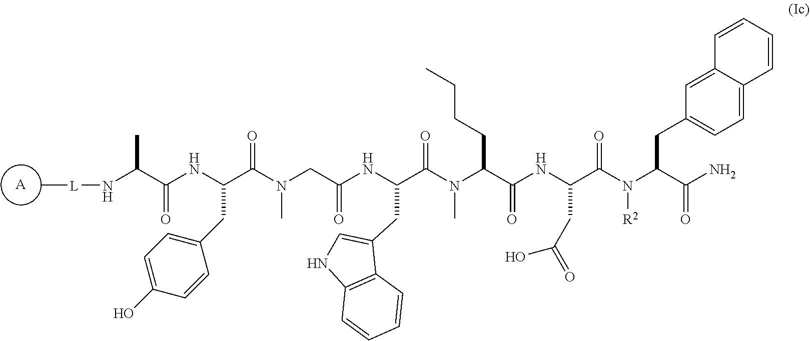
wherein R² is as defined in claim 1.

10. The compound, or the pharmaceutically acceptable salt thereof, according to claim 1, wherein R² is methyl.

11. The compound, or the pharmaceutically acceptable salt thereof, according to claim 1, wherein A is a radionuclide binding ligand selected from DOTA, DOTA-NHS-ester, p-SCN-Bn-DOTA, DOTAGA, DOTAGA-anhydride, CB-DO2A, TCMC, p-SCN-Bn-TCMC, 3p-C-DEPA, 3p-C-DEPA-NCS, p-NH₂-Bn-Oxo-DO3A, TETA, BAT, p-NH₂-Bn-TE3A, C-TETA, CB-TE2A, CB-TE1A1P, CB-TE2P, MM-TE2A, DM-TE2A, TE2A, Diamsar, SarAr, AmBaSar, BaBaSar, NOTA, p-SCN-Bn-NOTA, NODASA, NODAGA, NETA, NETA-monoamide, C-NE3TA-NCS, C-NETA-NCS, 3p-C-NETA, TACN-TM, DTPA,p-SCN-Bn-1B-DTPA,p-SCN-Bn-1B4M-DTPA, CHX-A″-DTPA,p-SCN-Bn-CHX-A″-DTPA, TRAP, AAZTA, NOPO, H₂dedpa, H₄octapa, H₂azapa, Hsdecapa, p-SCN-Bn-H₄octapa, HBED, HBED-CC, (HBED-CC)TFP, SHBED, BPCA, CP256, PCTA, p-SCN-Bn-PCTA, DFO, H₆phospha, p-SCN-Bn-DFO, HEHA, p-SCN-Bn-HEHA, PEPA, p-SCN-Bn-PEPA, Crown, MACROPA, MACROPA-NCS, pypa, py4pa, noneunpa, DOTAM, and derivatives thereof.

12. The compound, or the pharmaceutically acceptable salt thereof, according to claim 1, wherein A is DOTA.

13. The compound, or the pharmaceutically acceptable salt thereof, according to claim 1, further comprising a radionuclide complexed to the radionuclide binding ligand.

14. A pharmaceutical composition comprising a compound according to claim 13, or a pharmaceutically acceptable salt thereof.

15. A method for identifying CCK-2R positive cancer in a subject, comprising administering to the subject an effective amount of a compound according to claim 13, or a pharmaceutically acceptable salt thereof, and detection of the radionuclide.

16. The method according to claim 15, wherein the radionuclide is detected using positron emission tomography (PET).

17. The method according to claim 15, wherein the radionuclide is selected from carbon-11, nitrogen-13, oxygen-15, fluorine-18, scandium-44, copper-64, gallium-67, gallium-68, yttrium-86, zirconium-89, Technetium-99m, indium-111, iodine-124, iodine-125 and terbium-152.

18. A method of treating CCK-2R positive cancer in a subject in need thereof comprising administering to the subject an effective amount of compound according to claim 13, or a pharmaceutically acceptable salt thereof.

19. The method according to claim 18, wherein the CCK-2R positive cancer is selected from medullary thyroid carcinoma, somatostatin-2R negative neuroendocrine tumour, stromal ovarian cancer, or small cell lung cancer.

20. The method according to claim 18, wherein the radionuclide is selected from copper-67, yttrium-90, iodine-131, terbium-149, terbium-161, lutetium-177, rhenium-186, rhenium-188, bismuth-212, bismuth-213, astatine-211, lead-212 and actinium-225.

21.-22. (canceled)


SEQUENCE LISTING (27 sequences)

SEQ ID NO: 1 - protein, 13 aa, synthetic construct
  SITE 1: note = D-glutamic acid
  SITE 2: note = D-glutamic acid
  SITE 3: note = D-alanine
  SITE 4: note = D-glutamic acid
  SITE 5: note = D-glutamic acid
  SITE 6: note = D-glutamic acid
  SITE 9: note = X = MeGly
  SITE 11: note = X = N-methylnorleucine
  SITE 13: note = X = 3-(1-naphthyl)alanine
  SITE 13: note = amidated C-terminal
EEAEEEAYXWXDX

SEQ ID NO: 2 - protein, 13 aa, synthetic construct
  SITE 1: note = D-glutamic acid
  SITE 2: note = D-glutamic acid
  SITE 3: note = D-alanine
  SITE 4: note = D-glutamic acid
  SITE 5: note = D-glutamic acid
  SITE 6: note = D-glutamic acid
  SITE 9: note = X = MeGly
  SITE 11: note = X = N-methylnorleucine
  SITE 13: note = X = N-methylphenylalanine
  SITE 13: note = amidated C-terminal
EEAEEEAYXWXDX

SEQ ID NO: 3 - protein, 13 aa, synthetic construct
  SITE 1: note = D-glutamic acid
  SITE 2: note = D-glutamic acid
  SITE 3: note = D-alanine
  SITE 4: note = D-glutamic acid
  SITE 5: note = D-glutamic acid
  SITE 6: note = D-glutamic acid
  SITE 9: note = D-glutamic acid
  SITE 11: note = X = N-methylnorleucine
  SITE 13: note = amidated C-terminal
EEAEEEAYXWXDF

SEQ ID NO: 4 - protein, 13 aa, synthetic construct
  SITE 1: note = D-glutamic acid
  SITE 2: note = D-glutamic acid
  SITE 3: note = D-alanine
  SITE 4: note = D-glutamic acid
  SITE 5: note = D-glutamic acid
  SITE 6: note = D-glutamic acid
  SITE 9: note = X = MeGly
  SITE 11: note = X = N-methylnorleucine
  SITE 13: note = X = N-methyl-3-(1-naphthyl)alanine
  SITE 13: note = amidated C-terminal
EEAEEEAYXWXDX

SEQ ID NO: 5 - protein, 13 aa, synthetic construct
  SITE 1: note = D-glutamic acid
  SITE 2: note = D-glutamic acid
  SITE 3: note = D-alanine
  SITE 4: note = D-glutamic acid
  SITE 5: note = D-glutamic acid
  SITE 6: note = D-glutamic acid
  SITE 9: note = X = MeGly
  SITE 11: note = X = N-methylnorleucine
  SITE 13: note = X = 4-fluorophenylalanine
  SITE 13: note = amidated C-terminal
EEAEEEAYXWXDX

SEQ ID NO: 6 - protein, 13 aa, synthetic construct
  SITE 1: note = D-glutamic acid
  SITE 2: note = D-glutamic acid
  SITE 3: note = D-alanine
  SITE 4: note = D-glutamic acid
  SITE 5: note = D-glutamic acid
  SITE 6: note = D-glutamic acid
  SITE 9: note = X = MeGly
  SITE 11: note = X = N-methylnorleucine
  SITE 13: note = X = 3,4-difluorophenylalanine
  SITE 13: note = amidated C-terminal
EEAEEEAYXWXDX

SEQ ID NO: 7 - protein, 13 aa, synthetic construct
  SITE 1: note = D-glutamic acid
  SITE 2: note = D-glutamic acid
  SITE 3: note = D-alanine
  SITE 4: note = D-glutamic acid
  SITE 5: note = D-glutamic acid
  SITE 6: note = D-glutamic acid
  SITE 9: note = X = MeGly
  SITE 11: note = X = N-methylnorleucine
  SITE 13: note = X = 3,4,5-trifluorophenylalanine
  SITE 13: note = amidated C-terminal
EEAEEEAYXWXDX

SEQ ID NO: 8 - protein, 13 aa, synthetic construct
  SITE 1: note = D-glutamic acid
  SITE 2: note = D-glutamic acid
  SITE 3: note = D-alanine
  SITE 4: note = D-glutamic acid
  SITE 5: note = D-glutamic acid
  SITE 6: note = D-glutamic acid
  SITE 9: note = X = MeGly
  SITE 11: note = X = N-methylnorleucine
  SITE 13: note = X = 4-aminophenylalanine
  SITE 13: note = amidated C-terminal
EEAEEEAYXWXDX

SEQ ID NO: 9 - protein, 13 aa, synthetic construct
  SITE 1: note = D-glutamic acid
  SITE 2: note = D-glutamic acid
  SITE 3: note = D-alanine
  SITE 4: note = D-glutamic acid
  SITE 5: note = D-glutamic acid
  SITE 6: note = D-glutamic acid
  SITE 9: note = X = MeGly
  SITE 11: note = N-methylnorleucine
  SITE 13: note = X = 3-(2-naphthyl)alanine
  SITE 13: note = amidated C-terminal
EEAEEEAYXWXDX

SEQ ID NO: 10 - protein, 13 aa, synthetic construct
  SITE 1: note = D-glutamic acid
  SITE 2: note = D-glutamic acid
  SITE 3: note = D-alanine
  SITE 4: note = D-glutamic acid
  SITE 5: note = D-glutamic acid
  SITE 6: note = D-glutamic acid
  SITE 9: note = X = MeGly
  SITE 11: note = X = N-methylnorleucine
  SITE 13: note = X = 4-cyanophenylalanine
  SITE 13: note = amidated C-terminal
EEAEEEAYXWXDX

SEQ ID NO: 11 - protein, 13 aa, synthetic construct
  SITE 1: note = D-glutamic acid
  SITE 2: note = D-glutamic acid
  SITE 3: note = D-alanine
  SITE 4: note = D-glutamic acid
  SITE 5: note = D-glutamic acid
  SITE 6: note = D-glutamic acid
  SITE 9: note = X = MeGly
  SITE 11: note = X = N-methylnorleucine
  SITE 13: note = X = 4-trifluoromethylphenylalanine
  SITE 13: note = amidated C-terminal
EEAEEEAYXWXDX

SEQ ID NO: 12 - protein, 13 aa, synthetic construct
  SITE 1: note = D-glutamic acid
  SITE 2: note = D-glutamic acid
  SITE 3: note = D-alanine
  SITE 4: note = D-glutamic acid
  SITE 5: note = D-glutamic acid
  SITE 6: note = D-glutamic acid
  SITE 9: note = X = MeGly
  SITE 11: note = X = N-methylnorleucine
  SITE 13: note = amidated C-terminal
EEAEEEAYXWXDY

SEQ ID NO: 13 - protein, 13 aa, synthetic construct
  SITE 1: note = D-glutamic acid
  SITE 2: note = D-glutamic acid
  SITE 3: note = D-alanine
  SITE 4: note = D-glutamic acid
  SITE 5: note = D-glutamic acid
  SITE 6: note = D-glutamic acid
  SITE 9: note = X = MeGly
  SITE 11: note = X = N-methylnorleucine
  SITE 13: note = X = 2,3,4,5,6-pentafluorophenylalanine
  SITE 13: note = amidated C-terminal
EEAEEEAYXWXDX

SEQ ID NO: 14 - protein, 13 aa, synthetic construct
  SITE 1: note = D-glutamic acid
  SITE 2: note = D-glutamic acid
  SITE 3: note = D-alanine
  SITE 4: note = D-glutamic acid
  SITE 5: note = D-glutamic acid
  SITE 6: note = D-glutamic acid
  SITE 9: note = X = MeGly
  SITE 11: note = X = N-methylnorleucine
  SITE 13: note = X = 3-fluorophenylalanine
  SITE 13: note = amidated C-terminal
EEAEEEAYXWXDX

SEQ ID NO: 15 - protein, 13 aa, synthetic construct
  SITE 1: note = D-glutamic acid
  SITE 2: note = D-glutamic acid
  SITE 3: note = D-alanine
  SITE 4: note = D-glutamic acid
  SITE 5: note = D-glutamic acid
  SITE 6: note = D-glutamic acid
  SITE 9: note = X = MeGly
  SITE 11: note = X = N-methylnorleucine
  SITE 13: note = X = 4-acetylphenylalanine
  SITE 13: note = amidated C-terminal
EEAEEEAYXWXDX

SEQ ID NO: 16 - protein, 13 aa, synthetic construct
  SITE 1: note = D-alanine
  SITE 2: note = D-glutamic acid
  SITE 3: note = D-glutamic acid
  SITE 5: note = D-glutamic acid
  SITE 4: note = D-glutamic acid
  SITE 6: note = D-glutamic acid
  SITE 9: note = X = MeGly
  SITE 11: note = X = N-methylnorleucine
  SITE 13: note = X = 3-(1-naphthyl)alanine
  SITE 13: note = amidated C-terminal
AEEEEEAYXWXDX

SEQ ID NO: 17 - protein, 13 aa, synthetic construct
  SITE 1: note = D-glutamic acid
  SITE 2: note = D-alanine
  SITE 3: note = D-glutamic acid
  SITE 4: note = D-glutamic acid
  SITE 5: note = D-glutamic acid
  SITE 6: note = D-glutamic acid
  SITE 9: note = X = MeGly
  SITE 11: note = X = N-methylnorleucine
  SITE 13: note = X = 3-(1-naphthyl)alanine
  SITE 13: note = amidated C-terminal
EAEEEEAYXWXDX

SEQ ID NO: 18 - protein, 13 aa, synthetic construct
  SITE 1: note = D-glutamic acid
  SITE 2: note = D-glutamic acid
  SITE 3: note = D-glutamic acid
  SITE 4: note = D-alanine
  SITE 5: note = D-glutamic acid
  SITE 6: note = D-glutamic acid
  SITE 9: note = X = MeGly
  SITE 11: note = X = N-methylnorleucine
  SITE 13: note = X = 3-(1-naphthyl)alanine
  SITE 13: note = amidated C-terminal
EEEAEEAYXWXDX

SEQ ID NO: 19 - protein, 13 aa, synthetic construct
  SITE 1: note = D-glutamic acid
  SITE 2: note = D-glutamic acid
  SITE 3: note = D-glutamic acid
  SITE 4: note = D-glutamic acid
  SITE 5: note = D-alanine
  SITE 6: note = D-glutamic acid
  SITE 9: note = X = MeGly
  SITE 11: note = X = N-methylnorleucine
  SITE 13: note = X = 3-(1-naphthyl)alanine
  SITE 13: note = amidated C-terminal
EEEEAEAYXWXDX

SEQ ID NO: 20 - protein, 13 aa, synthetic construct
  SITE 1: note = D-glutamic acid
  SITE 2: note = D-glutamic acid
  SITE 3: note = D-glutamic acid
  SITE 4: note = D-glutamic acid
  SITE 5: note = D-glutamic acid
  SITE 6: note = D-alanine
  SITE 9: note = X = MeGly
  SITE 11: note = X = N-methylnorleucine
  SITE 13: note = X = 3-(1-naphthyl)alanine
  SITE 13: note = amidated C-terminal
EEEEEAAYXWXDX

SEQ ID NO: 21 - protein, 13 aa, synthetic construct
  SITE 1: note = D-glutamic acid
  SITE 2: note = D-glutamic acid
  SITE 3: note = D-glutamic acid
  SITE 4: note = D-glutamic acid
  SITE 5: note = D-glutamic acid
  SITE 6: note = D-glutamic acid
  SITE 9: note = X = MeGly
  SITE 11: note = X = N-methylnorleucine
  SITE 13: note = X = 3-(1-naphthyl)alanine
  SITE 13: note = amidated C-terminal
EEEEEEAYXWXDX

SEQ ID NO: 22 - protein, 13 aa, synthetic construct
  SITE 1: note = D-glutamic acid
  SITE 2: note = D-glutamic acid
  SITE 3: note = D-glutamic acid
  SITE 4: note = D-glutamic acid
  SITE 5: note = D-glutamic acid
  SITE 6: note = D-glutamic acid
  SITE 9: note = X = MeGly
  SITE 11: note = X = N-methylnorleucine
  SITE 13: note = X = 4-fluorophenylalanine
  SITE 13: note = amidated C-terminal
EEEEEEAYXWXDX

SEQ ID NO: 23 - protein, 13 aa, synthetic construct
  SITE 1: note = D-glutamic acid
  SITE 2: note = D-glutamic acid
  SITE 3: note = D-glutamic acid
  SITE 4: note = D-glutamic acid
  SITE 5: note = D-glutamic acid
  SITE 6: note = D-glutamic acid
  SITE 9: note = X = MeGly
  SITE 11: note = X = N-methylnorleucine
  SITE 13: note = X = 3,4-difluorophenylalanine
  SITE 13: note = amidated C-terminal
EEEEEEAYXWXDX

SEQ ID NO: 24 - protein, 13 aa, synthetic construct
  SITE 1: note = D-glutamic acid
  SITE 2: note = D-glutamic acid
  SITE 3: note = D-glutamic acid
  SITE 4: note = D-glutamic acid
  SITE 5: note = D-glutamic acid
  SITE 6: note = D-glutamic acid
  SITE 9: note = X = MeGly
  SITE 11: note = X = N-methylnorleucine
  SITE 13: note = X = 3,4,5-trifluorophenylalanine
  SITE 13: note = amidated C-terminal
EEEEEEAYXWXDX

SEQ ID NO: 25 - protein, 13 aa, synthetic construct
  SITE 1: note = D-glutamic acid
  SITE 2: note = D-glutamic acid
  SITE 3: note = D-glutamic acid
  SITE 4: note = D-glutamic acid
  SITE 5: note = D-glutamic acid
  SITE 6: note = D-glutamic acid
  SITE 9: note = X = MeGly
  SITE 11: note = X = N-methylnorleucine
  SITE 13: note = X = 2,3,4,5,6-pentafluorophenylalanine
  SITE 13: note = amidated C-terminal
EEEEEEAYXWXDX

SEQ ID NO: 26 - protein, 13 aa, synthetic construct
  SITE 1: note = D-glutamic acid
  SITE 2: note = D-glutamic acid
  SITE 3: note = D-glutamic acid
  SITE 4: note = D-glutamic acid
  SITE 5: note = D-glutamic acid
  SITE 6: note = D-glutamic acid
  SITE 9: note = X = MeGly
  SITE 11: note = X = N-methylnorleucine
  SITE 13: note = X = 4-aminophenylalanine
  SITE 13: note = amidated C-terminal
EEEEEEAYXWXDX

SEQ ID NO: 27 - protein, 13 aa, synthetic construct
  SITE 1: note = D-glutamic acid
  SITE 2: note = D-glutamic acid
  SITE 3: note = D-glutamic acid
  SITE 4: note = D-glutamic acid
  SITE 5: note = D-glutamic acid
  SITE 6: note = D-glutamic acid
  SITE 9: note = X = MeGly
  SITE 11: note = X = N-methylnorleucine
  SITE 13: note = X = 3-(2-naphthyl)alanine
  SITE 13: note = amidated C-terminal
EEEEEEAYXWXDX